(19) United States
(12) Patent Application Publication
(10) Pub. No.: US 2023/0174073 A1
(43) Pub. Date: Jun. 8, 2023

(54) DISEASE-CONDITION ASSESSMENT DEVICE, DISEASE-CONDITION ASSESSMENT METHOD, PROGRAM FOR DISEASE-CONDITION ASSESSMENT DEVICE, AND DISEASE-CONDITION ASSESSMENT SYSTEM

(71) Applicants: NATIONAL INSTITUTE OF ADVANCED INDUSTRIAL SCIENCE AND TECHNOLOGY, Tokyo (JP); UNIVERSITY OF TSUKUBA, Tsukuba-shi, Ibaraki (JP); YAZAKI CORPORATION, Tokyo (JP); SUBARU CORPORATION, Tokyo (JP); MITSUBISHI ELECTRIC CORPORATION, Tokyo (JP)

(72) Inventors: Hidehiko KOMINE, Ibaraki (JP); Satoshi KITAZAKI, Ibaraki (JP); motoyuki AKAKATSU, Ibaraki (JP); KEI ISHII, Ibaraki (JP); Hideo TSURUSHIMA, Ibaraki (JP); Motoki SHINO, Chiba (JP)

(21) Appl. No.: 17/995,506

(22) PCT Filed: Mar. 23, 2021
(86) PCT No.: PCT/JP2021/011933

§ 371 Date: Oct. 5, 2022

(30) Foreign Application Priority Data
Apr. 8, 2020 (JP) 2020069452

(51) Int. Cl. B60W 40/08; A61B 5/00; G06V 20/59

(52) U.S. Cl. CPC B60W 40/08; A61B 5/4094; G06V 20/597; B60W 2540/221; B60W 2540/225; B60W 2420/42; B60W 2540/223

(57) ABSTRACT

Provided are a disease-condition assessment device for being capable of assessing an epileptic condition of a subject, a disease-condition assessment method, a program for disease-condition assessment device, and a disease-condition assessment system. The gaze data indicating a subject's gazing point measured when the subject is driving a vehicle is acquired, the driving-characteristic data indicating driving characteristics of the subject for the vehicle is acquired), and the epileptic condition of the subject is assessed depending on the relationship between the gaze data and the driving-characteristic data.

### TECHNICAL FIELD

The present invention relates to techniques for a disease-condition assessment device for assessing an epileptic condition of a subject, a disease-condition assessment method, a program for the disease-condition assessment device, and a disease-condition assessment system.

### BACKGROUND ART

Various assessment systems have been developed to detect the condition of a driver driving a vehicle. For example, Patent Literature 1 discloses a safe driving support system in which a device that measures the driving ability and health information of a vehicle driver and transmits the driving ability check results and the health information check results to a server is installed at a plurality of points along the vehicle travel route, and the server evaluates the driving risk level of the vehicle driver based on the driving ability check results, the health information check results, and the evaluation criteria and diagnoses an increase in the driving risk level of the vehicle driver by comparing the driving ability check results and the health information check results over time.

### CITATION LIST

### Patent Literature

Patent Literature 1: JP 2015-141536 A

### SUMMARY OF INVENTION

### Problem to be Solved by the Invention

However, in the prior art like Patent Literature 1, in order to measure health condition, special equipment such as breath gas component measuring devices were required, and the prior art simply assessed the driving risk level in combinations with exercise capacity without direct relation to a particular disease. Therefore, it was difficult to assess epileptic condition in a simple way.

Hence, it is an object of the present invention to provide, for example, a disease-condition assessment device, etc., for assessing the epileptic condition.

### Means for Solving the Problem

To solve the above problem, the invention according to claim 1 includes: gaze data acquiring means for acquiring gaze data indicating a subject's gazing point measured when the subject is driving a vehicle; driving-characteristic data acquiring means for acquiring driving-characteristic data indicating driving characteristics of the subject for the vehicle; and disease-condition assessment means for assessing an epileptic condition of the subject depending on the relationship between the gaze data and the driving-characteristic data.

The invention according to claim 8 includes: a step in which gaze data acquiring means acquires gaze data indicating a subject's gazing point measured when the subject is driving a vehicle; a step in which driving-characteristic data acquiring means acquires driving-characteristic data indicating driving characteristics of the subject for the vehicle; and a step in which disease-condition assessment means is capable of assessing an epileptic condition of the subject depending on the relationship between the gaze data and the driving-characteristic data.

The invention according to claim 9 causes a computer to function as: gaze data acquiring means for acquiring gaze data indicating a subject's gazing point measured when the subject is driving a vehicle; driving-characteristic data acquiring means for acquiring driving-characteristic data indicating driving characteristics of the subject for the vehicle; and disease-condition assessment means for being capable of assessing an epileptic condition of the subject depending on the relationship between the gaze data and the driving-characteristic data.

The invention according to claim 10 is a disease-condition assessment system including a terminal device that collects data related to a subject driving a vehicle and a disease-condition assessment device for assessing an epileptic condition of the subject based on the data related to the subject, and the disease-condition assessment device includes: gaze data acquiring means for acquiring gaze data indicating a subject's gazing point measured when the subject is driving a vehicle; driving-characteristic data acquiring means for acquiring driving-characteristic data indicating driving characteristics of the subject for the vehicle; and disease-condition assessment means for being capable of assessing an epileptic condition of the subject depending on the relationship between the gaze data and the driving-characteristic data.

The invention according to claim 11 includes: seat pressure distribution acquiring means for acquiring a data of a seat pressure distribution on a seating surface where the subject is seated in the vehicle; pressure distribution calculation means for calculating a size of the seat pressure distribution; and disease-condition assessment means for assessing an epileptic condition of the subject depending on a change in the size of the seat pressure distribution.

The invention according to claim 12 includes: a step in which seat pressure distribution acquiring means acquires data of a seat pressure distribution on a seating surface where the subject is seated in the vehicle; a step in which pressure distribution calculation means calculates a size of the seat pressure distribution; and a step in which disease-condition assessment means is capable of assessing an epileptic condition of the subject depending on a change in the size of the seat pressure distribution.

The invention according to claim 13 causes a computer to function as: seat pressure distribution acquiring means for acquiring data of a seat pressure distribution on a seating surface where the subject is seated in the vehicle; pressure distribution calculation means for calculating a size of the seat pressure distribution; and disease-condition assessment means for being capable of assessing an epileptic condition of the subject depending on a change in the size of the seat pressure distribution.

The invention according to claim 14 is a disease-condition assessment system including a terminal device that collects data related to a subject driving a vehicle and a disease-condition assessment device for assessing an epileptic condition of the subject based on the data related to the subject, and the system includes: seat pressure distribution acquiring means for acquiring data of a seat pressure distribution on a seating surface where the subject is seated in the vehicle; pressure distribution calculation means for calculating a size of the seat pressure distribution; and disease-condition assessment means for being capable of assessing an epileptic condition of the subject depending on a change in the size of the seat pressure distribution.

The invention according to claim 15 includes: gaze data acquiring means for acquiring gaze data indicating a subject's gazing point measured when the subject is driving a vehicle; and disease-condition assessment means for assessing an epileptic condition of the subject depending on duration while the gazing point indicated by the gaze data is out of the center part of a visual field in the traveling direction of the vehicle.

The invention according to claim 16 includes: a step in which gaze data acquiring means acquires gaze data indicating a subject's gazing point measured when the subject is driving a vehicle; and a step in which disease-condition assessment means is capable of assessing an epileptic condition of the subject depending on duration while the gazing point indicated by the gaze data is out of the center part of a visual field in the traveling direction of the vehicle.

The invention according to claim 17 causes a computer to function as: gaze data acquiring means for acquiring gaze data indicating a subject's gazing point measured when the subject is driving a vehicle; and disease-condition assessment means for being capable of assessing an epileptic condition of the subject depending on duration while the gazing point indicated by the gaze data is out of the center part of a visual field in the traveling direction of the vehicle.

The invention according to claim 18 is a disease-condition assessment system including a terminal device that collects data related to a subject driving a vehicle and a disease-condition assessment device for assessing an epileptic condition of the subject based on the data related to the subject, and the disease-condition assessment device includes: gaze data acquiring means for acquiring gaze data indicating a subject's gazing point measured when the subject is driving a vehicle; and disease-condition assessment means for being capable of assessing an epileptic condition of the subject depending on duration while the gazing point indicated by the gaze data is out of the center part of a visual field in the traveling direction of the vehicle.

### Effect of the Invention

According to the present invention, by assessing a disease condition of subject T, such as epilepsy, depending on the relationship between the gaze data indicating the subject's gazing point measured when the subject is driving a vehicle and the driving-characteristic data indicating driving characteristics of the subject for the vehicle, it is possible to assess the epileptic condition of the subject from data that is easy to measure, such as the gaze data and the driving-characteristic data, without special equipment.

### BRIEF DESCRIPTION OF DRAWINGS

FIG. 1 is a schematic diagram showing an example of overview configuration of a disease-condition assessment system according to an embodiment.

FIG. 2 is a schematic diagram showing an example of appearance of a subject steering a vehicle.

FIG. 3 is a block diagram schematically showing an example configuration of an information processing server device in FIG. 1.

FIG. 4 is a diagram showing an example of data stored in a subject information database in FIG. 3.

FIG. 5 is a diagram showing an example of data stored in an operation-quantity database in FIG. 3.

FIG. 6 is a diagram showing an example of data stored in a movement-quantity database in FIG. 3.

FIG. 7 is a diagram showing an example of data stored in a subject sensing database in FIG. 3.

FIG. 8 is a diagram showing an example of data stored in a disease assessment database in FIG. 3.

FIG. 9A is a graph showing an example of gaze data and steering angle data during driving.

FIG. 9B is a graph showing an example of gaze data and steering angle data during driving.

FIG. 9C is a graph showing an example of gaze data and steering angle data during driving.

FIG. 10A is a graph showing an example of gaze data and steering torque data during driving.

FIG. 10B is a graph showing an example of gaze data and steering torque data during driving.

FIG. 10C is a graph showing an example of gaze data and steering torque data during driving.

FIG. 11A is a graph showing an example of gaze data and lateral acceleration data during driving.

FIG. 11B is a graph showing an example of gaze data and lateral acceleration data during driving.

FIG. 11C is a graph showing an example of gaze data and lateral acceleration data during driving.

FIG. 12A is a graph showing an example of the degree of dissociation between gaze movement and steering angle, and the elapsed time after the dissociation.

FIG. 12B is a graph showing an example of the degree of dissociation between the gaze movement and the steering torque, and the elapsed time after the dissociation.

FIG. 12C is a graph showing an example of the degree of dissociation between the gaze movement and the lateral acceleration, and the elapsed time after the dissociation.

FIG. 13A is a graph showing an example of operation-related data.

FIG. 13B is a graph showing an example of operation-related data.

FIG. 13C is a graph showing an example of movement-related data.

FIG. 14A is a graph showing an example of duration that the gaze point was continuously located outside the center part.

FIG. 14B is a graph showing an example of duration that the gaze point was continuously located outside the center part.

FIG. 14C is a graph showing an example of duration that the gaze point was continuously located outside the center part.

FIG. 14D is a graph showing an example of duration that the gaze point was continuously located outside the center part.

FIG. 15A is a graph showing an example of duration that the gaze point was continuously located outside the center part.

FIG. 15B is a graph showing an example of duration that the gaze point was continuously located outside the center part.

FIG. 15C is a graph showing an example of duration that the gaze point was continuously located outside the center part.

FIG. 15D is a graph showing an example of duration that the gaze point was continuously located outside the center part.

FIG. 16A is a graph showing an example of duration that the gaze point was continuously located outside the center part.

FIG. 16B is a graph showing an example of duration that the gaze point was continuously located outside the center part.

FIG. 17A is a graph showing an example of average duration time that the gaze point was continuously located outside the center part.

FIG. 17B is a graph showing an example of average duration time that the gaze point was continuously located outside the center part.

FIG. 18A is a graph showing an example of rate of duration time that the gaze point was continuously located outside the center part.

FIG. 18B is a graph showing an example of rate of duration time that the gaze point was continuously located outside the center part.

FIG. 19 is a schematic diagram showing an example of data of seat pressure distribution.

FIG. 20 is a graph showing an example of time series of seat-pressure center data and area of seat pressure distribution.

FIG. 21 is a graph showing an example of time series of seat-pressure center data and area of seat pressure distribution.

FIG. 22 is a block diagram schematically showing an example configuration of a mobile terminal device in FIG. 1.

FIG. 23 is a block diagram schematically showing an example configuration of an in-vehicle terminal device in FIG. 1.

FIG. 24 is a flowchart showing an operation example of data collection.

FIG. 25 is a schematic diagram showing an example of a road on which the vehicle traveled.

FIG. 26 is a flowchart showing an operation example of assessing the disease condition.

FIG. 27 is a schematic diagram showing an example of a road on which the vehicle traveled.

### DESCRIPTION OF EMBODIMENTS

The following describes an embodiment of the present invention with reference to the drawings. In the embodiment described below, the present invention is applied to a disease-condition assessment system.

[1. Configuration and Functional Overview of Disease-Condition Assessment System]

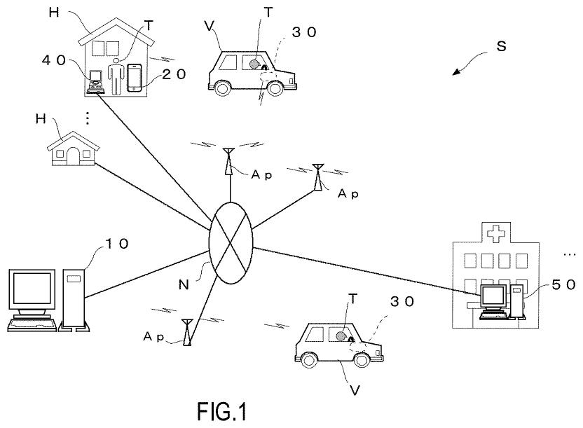

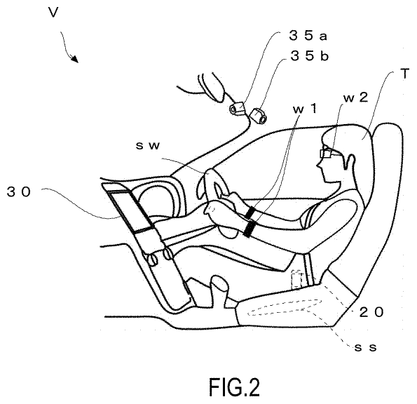

First, a configuration of disease-condition assessment system S according to an embodiment of the present invention is described using FIG. 1 and FIG. 2. FIG. 1 is a schematic diagram showing an example of overview configuration of a disease-condition assessment system S according to an embodiment. FIG. 2 is a schematic diagram showing an example of appearance of a subject T steering a vehicle V.

As shown in FIG. 1, a disease-condition assessment system S includes an information processing server device 10 (an example of a disease-condition assessment device), mobile terminal devices 20, in-vehicle terminal devices 30, home terminal devices 40, and medical institution server devices 50. The information processing server device 10 assesses disease condition of subjects T, such as epilepsy, from various data of each subject T driving vehicles V. Each mobile terminal device 20, which is carried by each subject T, transmits the physiological data of the subject T to the information processing server device 10. Each in-vehicle terminal device 30 collects data on the operation and movement of the vehicle V driven by the subject T from a plurality of sensors. Each home terminal devices 40 collects the physiological data, etc., when the subject T stays at the home H. The medical institution server device 50 is of a medical institution visited by subjects T.

Herein, examples of the vehicle V include, an automobile such as passenger car, taxis, hire, truck, trailer (including tractor alone) and bus, a motorcycle (side motorized motorcycle, trike, reverse trike), bicycle, electric cart, train like a train car.

Examples of subject T include, for example, a person driving the vehicle.

The information processing server device 10 is capable of exchanging data with each mobile terminal device 20, each in-vehicle terminal device 30, each home terminal device 40, and the medical institution server device 50 over a network N using communication protocols, such as TCP/IP. The network N includes, for example, the Internet.

In the network N, driving information provision server devices (not shown) are connected. The driving information provision server devices provide road information for road conditions such as traffic congestion, under construction, etc. The meteorological server devices (not shown) provide meteorological data to the information processing server device 10.

Incidentally, the network N may include, for example, a dedicated communication line, a mobile communication network, and a gateway. The network N may include access points Ap. For example, the mobile terminal device 20 and the in-vehicle terminal device 30 may be connectable to the network N through the access point Ap.

The information processing server device 10 has a function of a computer. The information processing server device 10 acquires driving-characteristic data indicating the driving characteristics of each subject T for the vehicle V, which are measured when each subject T is driving the vehicle V. An example of the driving-characteristic data includes, for example, operation-quantity data of each subject T operating the vehicle V and movement-quantity data of movement on each vehicle V. The information processing server device 10 acquires, for example, the operation-quantity data and the movement-quantity data from the in-vehicle terminal device 30.

In addition, the information processing server device 10 acquires data obtained by sensing for the subject T operating each vehicle V from the mobile terminal device 20 or the in-vehicle terminal device 30. For example, this sensing data is the gaze data indicating the gazing point of the subject T, measured when the subject T is driving the vehicle V, the data of the seat pressure distribution on the seating surface where the subject T operating each vehicle V sits, and the rotational data of the arm rotation of the subject T operating the vehicle V.

The information processing server device 10 acquires meteorological data from the meteorological server devices. The information processing server device 10 acquires road information from the driving information provision server devices.

Each mobile terminal device 20 has a function of a computer. The mobile terminal device 20 is, for example, a smartphone or a tablet terminal. The mobile terminal device 20 collects data from each of sensors that senses the subject T. As shown in FIG. 2, there is the mobile terminal device 20 somewhere in the vehicle V; for example, in the vehicle V, the subject T may have the portable terminal device 20 in their pocket, bag, etc.

Each in-vehicle terminal device 30 has a function of a computer. The in-vehicle terminal device 30 is, for example, a navigation device of the vehicle V. As shown in FIG. 2, the in-vehicle terminal device 30 is installed in the vehicle V which the subject T drives. The vehicle V is, for example, a vehicle owned by the subject T himself/herself, family, acquaintance or the company, or a vehicle rented.

As shown in FIG. 2, the subject T operates the vehicle V by the steering wheel sw, accelerator pedal (not shown), and brake pedal (not shown) of the vehicle V.

As shown in FIG. 2, the subject T wears each wristband-type wearable terminal device w1 on both arms, when measuring the rotation of both arms of the subject T. The subject T wears the eyeglass-type wearable terminal device w2, when measuring the gaze data indicating the position of the subject T's gazing point.

As shown in FIG. 2, the sheet sensor ss measures the seat pressure distribution on the seating surface where the subject T operating the vehicle V sits. The sheet sensor ss is a sheet-like sensor with pressure sensor elements distributed in two dimensions to measure the body pressure distribution. The sheet sensor ss measures the position and pressure on the surface of the sheet. Incidentally, the sheet sensor ss may be installed in the back of the seat.

The mobile terminal device 20 and the in-vehicle terminal device 30 can communicate by wireless communication. The wearable terminal devices w1, w2 can communicate with the mobile terminal device 20 and the in-vehicle terminal device 30 through wireless communication. The sheet sensor ss has an interface that allows communication with the outside. The sheet sensor ss can communicate with the mobile terminal device 20 and the in-vehicle terminal device 30 through wireless communication. The sheet sensor ss may be able to communicate with the in-vehicle terminal device 30 by wire.

Each home terminal device 40 has a function of a computer. The home terminal device 40 is installed in, for example, the home H of the subject T or his/her workplace. The home terminal device 40 is, for example, a personal computer. The mobile terminal device 20 and the home terminal device 40 can communicate by wireless communication.

The medical institution server device 50 has a function of a computer. The medical institution server device 50 is installed in, for example, medical institutions such as hospitals and core centers of regional medicine. The medical institution server device 50 has electronic medical records information which records information, such as result of consultation, examination order, results of examination, and medical checkup on the subject T.

Herein, the operation quantity is the quantity of some operation when the subject T drives the vehicle V. The operation quantity for the vehicle includes the steering angle of the vehicle V's steering wheel, the accelerator pedal application of the vehicle V's accelerator, the operation quantity of the brake pedal, etc. The operation-quantity may be a steering torque corresponding to the angular velocity of the steering wheel of the vehicle V and the angular acceleration of steering, calculated from the time differentiation of the steering angle data. The operation quantity may be any measurable data for the operation performance of the subject T.

In addition, the movement quantity is the quantity related to the movement of the vehicle V. The movement quantity of the vehicle includes fluctuation on the vehicle V, inter-vehicular distance to a vehicle in front, vehicle velocity, vehicle acceleration, position in the lane, etc. Acceleration includes acceleration in the traveling direction of vehicle V, lateral acceleration in the lateral direction for the traveling direction, etc. The movement quantity may be any data from which the movement state of the vehicle V by the driving of the subject T is measured.

The operation quantity and the movement quantity are the quantities that indicate the driving characteristics of the subject T for the vehicle V.

In addition, an example of the data obtained by sensing the subject T operating the vehicle V includes the gaze data indicating the position of the subject T's gazing point, measured when the subject T is driving the vehicle V, the data of the seat pressure distribution on the seating surface where the subject T operating the vehicle V sits, and the rotational data of the arm rotation of the subject T operating the vehicle V, etc.

Next, an example of the disease condition includes the disease severity, such as whether the disease condition for a predetermined disease is mild enough to drive or severe enough to prevent driving. An example of the disease condition includes types of diseases, such as epilepsy, stroke and epileptic seizure. The disease severity in case of epilepsy, may be a partial or generalized seizure. The disease severity in case of stroke, may be hemiplegia or diplegia, right-sided or left-sided paralysis.

Examples of the type of disease includes stroke, epileptic seizure, cardiovascular disease such as myocardial infarction, hypertension and arrhythmia, sleep apnea syndrome, dementia, a decrease in consciousness level due to diabetes.

In addition, the type of disease may include the type of symptom. Examples of symptoms include the degree of paralysis, palpitations, shortness of breath, constipation, fever, chills, diarrhea, numbness, pain, etc. The type of disease may include the disease severity. For example, in the case that the disease is stroke, it includes the levels of stroke without paralysis, with mild paralysis, and with paralysis. In the disease severity, another level ID from the disease ID may be used. For example, in the case of epileptic seizures, there is a difference between partial and generalized seizures.

In addition, an example of disease condition includes signs of sickness, risk of disease developing, and risk of symptom development for a predetermined disease. An example of disease condition may include the degree of signs of sickness, and the value of disease developing risk.

With regard to the determination of the sign of the symptom, it may be determined by a single index or combinations of indices. For example, palpitations may be determined only by heart rate, and shortness of breath may be uniquely determined by respiration rate (measured by thoracic movement, etc.). Moreover, blood pressure may be added to determine “effect due to shortness of breath”.

In addition, the type of disease may include the type of organ or organ system and the type of biological function. An example of disease condition may include levels of condition of each organ or organ system and levels of condition of each biological function (for example, digestive function, cardiovascular function, function of the nervous system, metabolic function and cognitive function, etc.). These levels may be levels corresponding to the specific numerical value such as blood test, considering age and body weight of the subject T, etc.

An example of disease condition may include probability of occurrence of a predetermined disease (developing risk). Instead of value of the probability, the predetermined disease may be “sickness A is less likely to develop”, “sickness A is apt to develop somewhat”, “sickness A is likely to develop”, “sickness A has become apparent”, etc.

An example of disease condition may include multiple sickness, for example, “sickness A and sickness B are likely to develop”, etc. The type of disease may be combinations of multiple diseases.

An example of disease condition may include “the risk of developing sickness A exceeded the first threshold”, “the risk of developing sickness B exceeded the first threshold”, “the risk of developing sickness A exceeded nth threshold”, and “the risk of developing sickness B exceeded nth threshold”.

An example of disease condition may include levels of physical condition. For example, regarding physical condition, it may be “health” and “poor physical condition,” or it is divided into levels, such as “good, somewhat good, somewhat abnormal, abnormal,” etc. In case of indicating the level of physical condition, disease name and the like may not be specified. Risks and levels are an example of quantitative assessment. In these cases, it is difficult to specify the type of disease, but it can be a preliminary condition.

As levels of physiological condition, it may be based on combinations of the number exceeding the threshold value and sickness exceeding the threshold value for each sickness.

In addition, examples of levels of physiological condition may include that value of the predetermined physiological data (or, driving-characteristic data of each subject T who is driving the vehicle V) “exceeded the first threshold”, . . . , “exceeded the nth threshold”. The level of the physiological condition may be based on combinations of multiple data.

In addition, not only captured in each individual physiological condition, but each of physiological conditions may be handled concurrently in a vector space (feature space of feature vectors). An index of each physiological condition may be captured in an n-dimensional vector space and may be handled like the level of the physiological condition by the positional relationship in the vector space.

[2. Configuration and Functions of Information Processing Server and Each Terminal Device]

(2.1 Configuration and Functions of Information Processing Server Device 10)

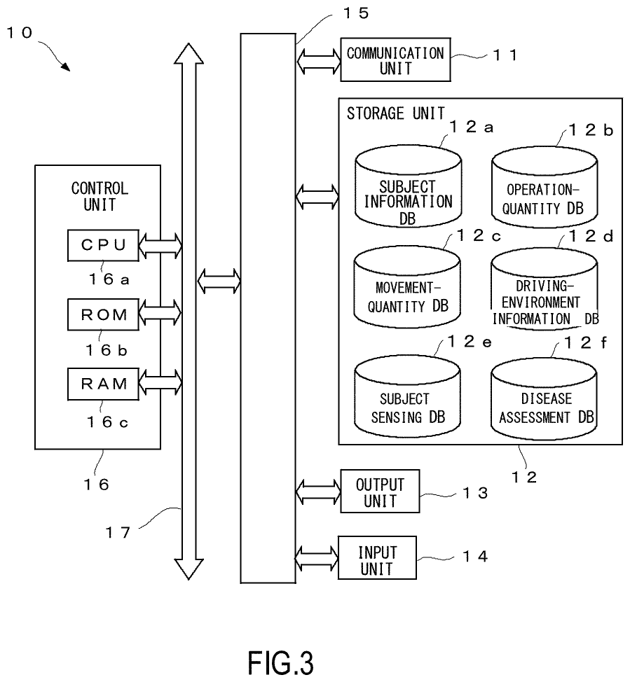

The following describes a configuration and functions of the information processing server device 10 using FIGS. 3 to 21.

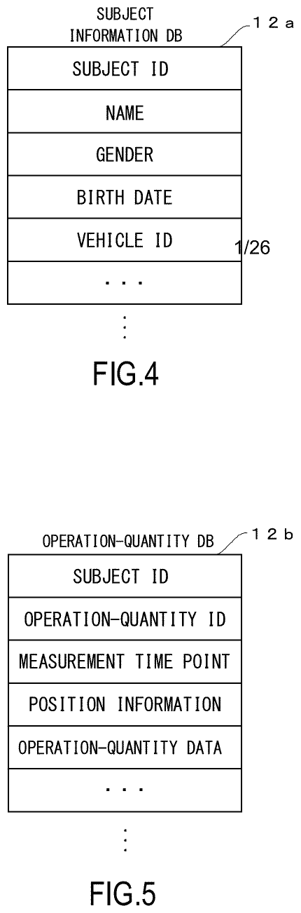

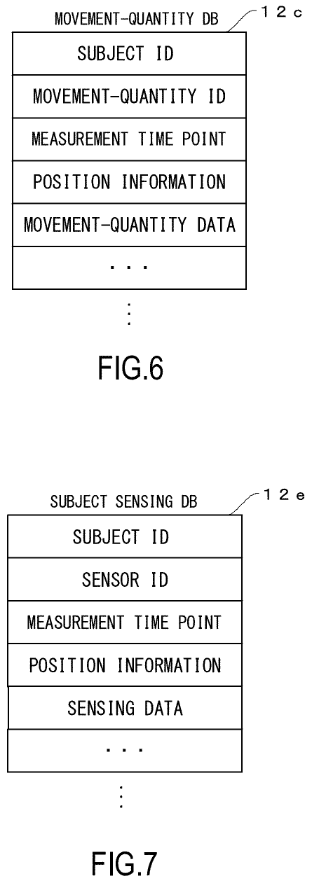

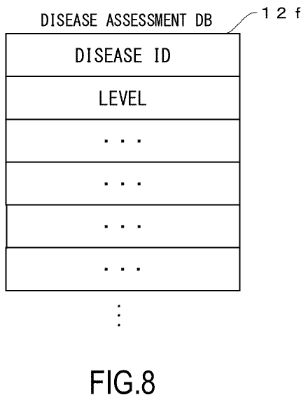

FIG. 3 is a block diagram schematically showing an example configuration of an information processing server device 10. FIG. 4 is a diagram showing an example of data stored in a subject information database. FIG. 5 is a diagram showing an example of data stored in an operation-quantity database. FIG. 6 is a diagram showing an example of data stored in a movement-quantity database. FIG. 7 is a diagram showing an example of data stored in a subject sensing database. FIG. 8 is a diagram showing an example of data stored in a disease assessment database.

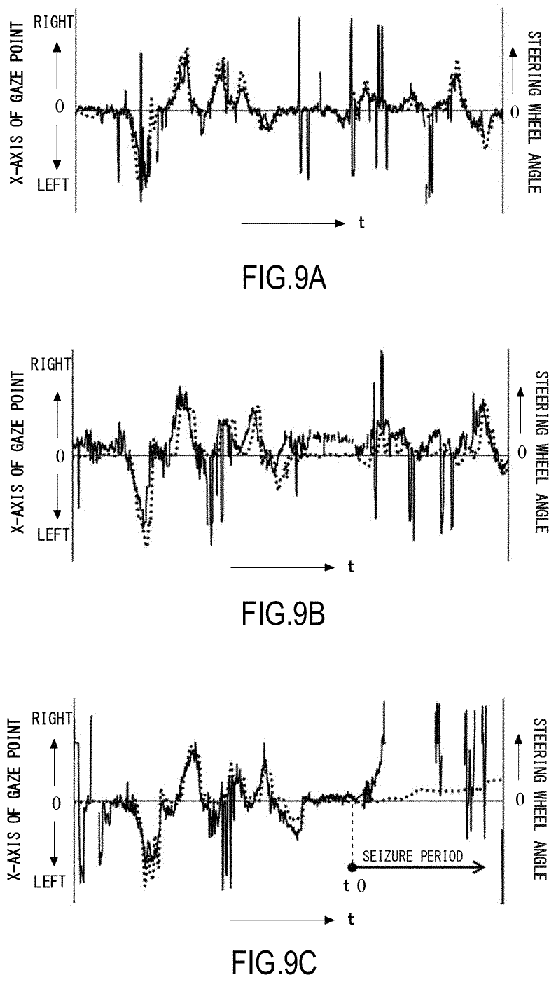

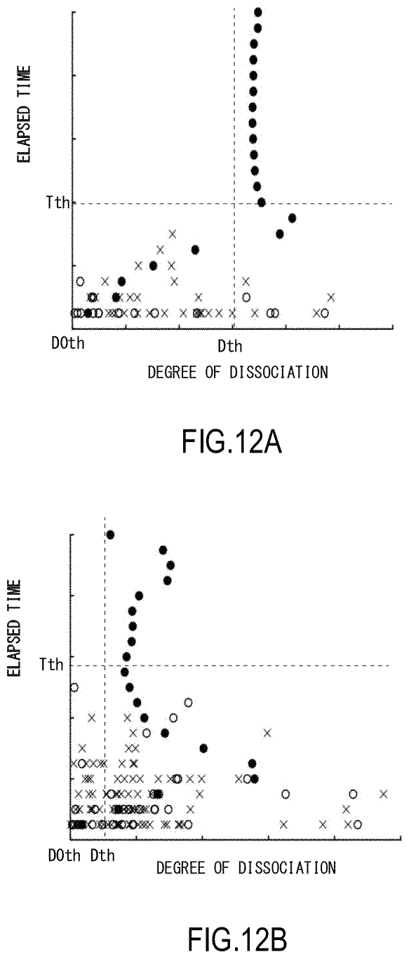

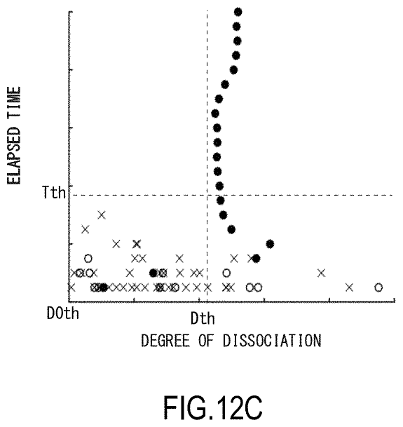

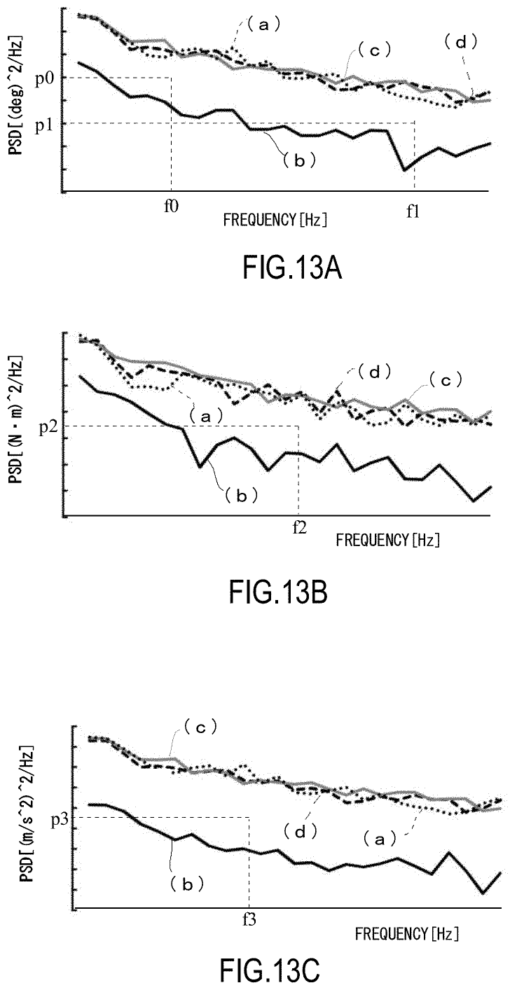

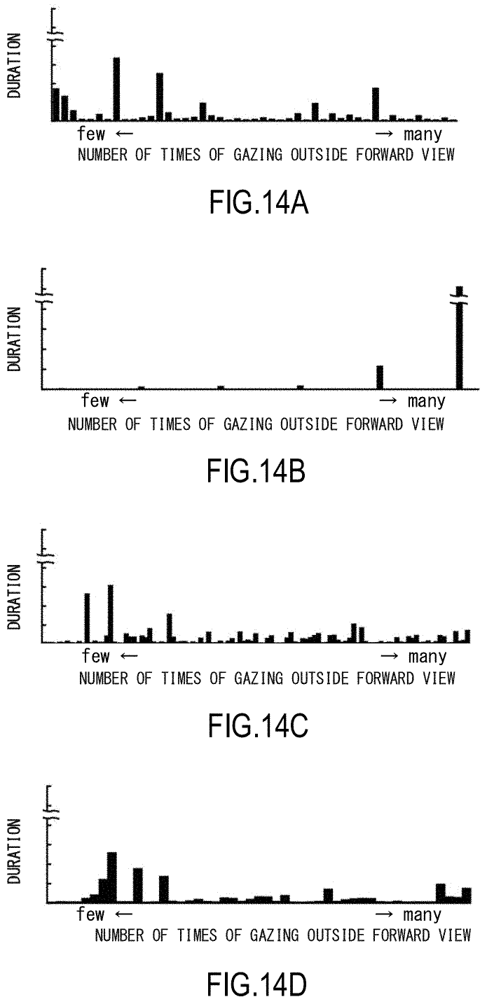

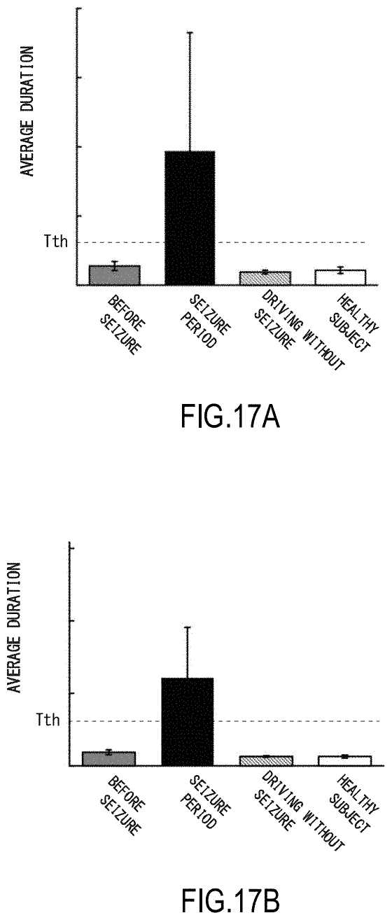

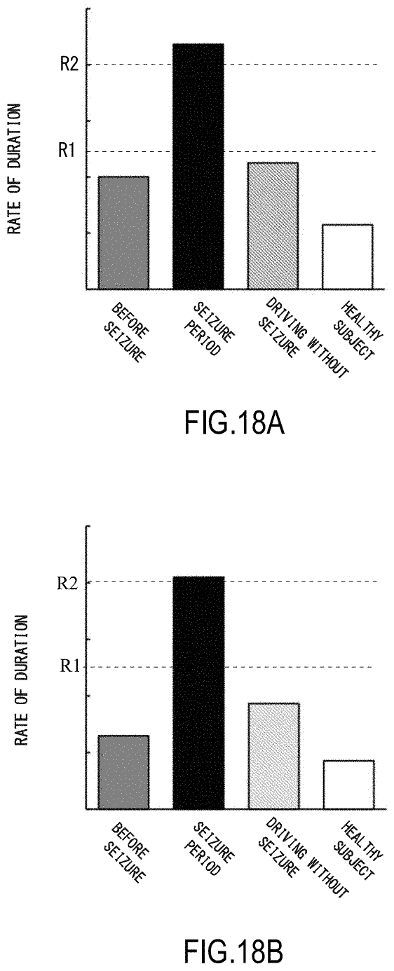

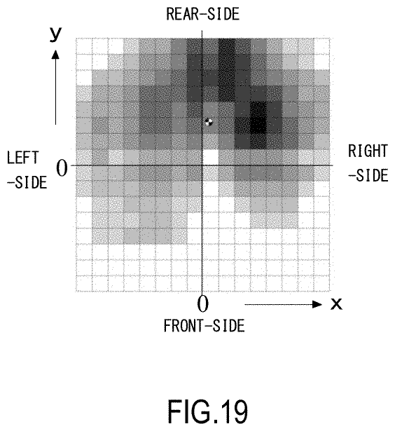

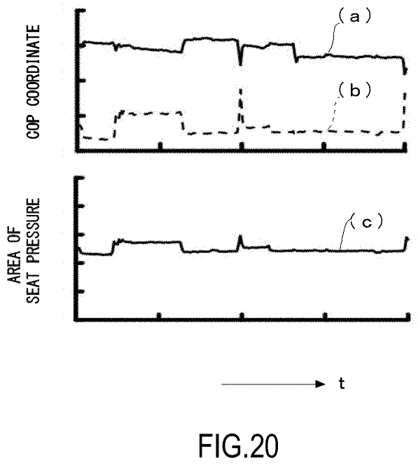

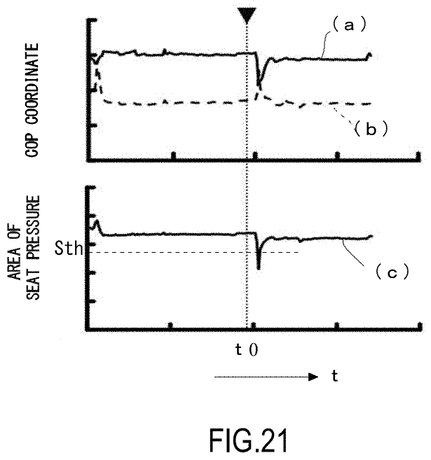

FIGS. 9A to 11C are graphs showing an example of gaze data and driving-characteristic data during driving. FIGS. 12A to 12C are graphs showing an example of the degree of dissociation between gaze movement and driving characteristic, and the elapsed time after the dissociation. FIGS. 13A to 13C are graphs showing an example of driving-characteristic data. FIGS. 14A to 16B are graphs showing an example of duration time that the gaze point was continuously located outside the center part. FIGS. 17A and 17B are graphs showing an example of average duration time that the gaze point was continuously located outside the center part. FIGS. 18A and 18B are graphs showing an example of rate of duration time that the gaze point was continuously located outside the center part. FIG. 19 is a schematic diagram showing an example of data of seat pressure distribution. FIGS. 20 and 21 are graphs showing an example of time series of seat-pressure center data and size of seat pressure distribution.

As shown in FIG. 3, the information processing server device 10 includes a communication unit 11, a storage unit 12, an output unit 13, an input unit 14, an input/output interface unit 15, and a control unit 16. The control unit 16 and the input/output interface unit 15 are connected electrically via a system bus 17. In addition, the information processing server device 10 has a clock function.

The communication unit 11 connects to the network N electrically or electromagnetically and controls the state of communications with, for example, the mobile terminal device 20.

The storage unit 12 includes, for example, hard disk drives or solid state drivers. The storage unit 12 stores data related to each vehicle V and the data obtained by sensing each subject T, etc. The storage unit 12 stores various programs, such as an operating system and server programs, and various files. Incidentally, the various programs may be obtained from, for example, another server device over the network N, or may be recorded in a recording medium and read via a drive device.

In the storage unit 12, a subject information database 12a (hereinafter, simply a “subject information DB 12a”), an operation-quantity database 12b (hereinafter, simply an “operation-quantity DB 12b”), a movement-quantity database 12c (hereinafter, simply a “movement-quantity DB 12c”), a driving-environment information database 12d (hereinafter, simply a “driving-environment information DB 12d”), a subject sensing database 12e (hereinafter, simply a “subject sensing DB 12e”), a disease assessment database 12f (hereinafter, simply a “disease assessment DB 12f”), and other databases are constructed.

The subject information DB 12a stores, for example, information concerning each of the subjects T. For example, as shown in FIG. 4, the subject information DB 12a stores the subject T's name, gender, date of birth, vehicle ID used by the subject T, etc. in association with the subject ID to identify each subject T.

The operation-quantity DB 12b stores the various operation-quantity data that the subject T on each vehicle V operated the vehicle V. For example, as shown in FIG. 5, the operation-quantity DB 12b stores measurement time points at which various operation quantities were measured while the subject T was driving the vehicle V, position information of the vehicle V, operation-quantity data, etc., in association with the subject ID and the operation-quantity ID for identifying each operation quantity. The operation-quantity ID corresponds to each operation quantity, such as the steering angle of the vehicle V's steering wheel sw, the accelerator pedal application of the vehicle V's accelerator, the operation quantity of the brake pedal, etc., and the IDs are assigned. Instead of the subject ID, it may be the vehicle ID that identifies each vehicle V. The position information of the vehicle V is latitude and longitude information or link information.

Herein, the accelerator pedal application is the quantity of accelerator pedal movement. The operation quantity of the accelerator pedal may be the number and frequency of sudden accelerations (the number and frequency of accelerations greater than or equal to a predetermined value).

The brake pedal application is the quantity of brake pedal movement. The operation quantity of the brake pedal may be the number and frequency of sudden braking (the number and frequency of decelerations greater than or equal to a predetermined value), time from when the brake is required to when the brake pedal is stepped on, the time from when the accelerator pedal is stepped off to when the brake pedal is stepped on, etc.

The movement-quantity DB 12c stores the movement-quantity data indicating the movement on each vehicle V driven by the subject T. For example, as shown in FIG. 6, the movement-quantity DB 12c stores measurement time points at which movement quantities were measured while the subject T was driving the vehicle V, position information of the vehicle V, movement-quantity data, etc., in association with the subject ID and the movement-quantity ID for identifying each movement quantity. The IDs are assigned corresponding to each movement quantity, such as the fluctuation on the vehicle V, the inter-vehicular distance to a vehicle in front, the vehicle's lateral acceleration, vehicle velocity, the acceleration in the traveling direction of vehicle, etc. Instead of the subject ID, it may be the vehicle ID that identifies each vehicle V.

The driving-environment information DB 12d stores map information and driving-environment information such as road attributes or types of roads, such as whether it is a highway or a general road, the degree of curve of the road, etc., and road information of road states, such as traffic congestion, under construction, etc.

The map information may also include link information. Herein, a link is a line segment of a road that connects nodes such as road intersections, change points of road structure, etc.

In addition, an example of the degree of curvature of a road includes, for example, the curvature of the curve of the road, the average curvature in a certain section of the road, and the percentage or number of roads with curvature above a predetermined value. An example of the degree of curvature of a road may include a road with more curves or less curves simply. An example of the degree of curvature of a road may include patterns of how the road curves. An example of the degree of curvature of a road may include road classification such as roads with a high degree of curves, like the Metropolitan Expressway in Tokyo, and roads with a relatively large number of straight sections. An example of the degree of curvature of a road may include classification of road type such as a general road, a highway, the Metropolitan Expressway in Tokyo, and a mountain road. It may include classification such as a standard highway with relatively few curves and highways with relatively many curves, like the Metropolitan Expressway in Tokyo. It may include classification such as highways with frequent branches, like Tokyo's Metropolitan Expressway, and those that do not. Furthermore, a collection of road sections with curvature within a predetermined range may be used as the classification of the road.

The driving-environment information DB 12d stores the degree of curvature of the road, the type of road, etc., in association with the road classification ID indicating the classification of the road.

In addition to the above, an example of the driving-environment information includes road information such as temporary stop place, one way road, two lane road, and road with median strip. An example of the driving-environment information includes, for example, whether the width of the road is narrow or wide, whether it is a usually used road or a road used for the first time, whether there are many or few pedestrians, whether vehicle traffic volume is high or low (even if it cannot say that it is congestion). In addition, an example of driving-environment information includes, for example, information that the road is dazzling with sunlight depending on the time zone, roads where drivers get nervous easily, roads where heart rate tends to rise, length of driving time, probability of occurrence of accidents in each location. The information on traffic congestion may include information on whether traffic was congested, time zone such as rush hour, infrastructure information such as road construction and accident. Incidentally, the information processing server device 10 acquires the latest road information from the driving information provision server devices. The information processing server device 10 may store past traffic congestion information.

Next, the subject sensing DB 12e stores the data of each subject T who is driving each vehicle V, sensed by various sensors. For example, as shown in FIG. 7, the subject sensing DB 12e stores the measurement time points at which the subject T was measured by each sensor, location information of the vehicle V, sensing data, etc., in association with the subject ID and the sensor ID for identifying each sensor.

An example of the data obtained by sensing for the subject T operating the vehicle V includes the gaze data indicating the position of the subject T's gazing point, measured when the subject T is driving the vehicle V, the data of the seat pressure distribution on the seating surface where the subject T operating the vehicle V sits, and the rotational data of the arm rotation of the subject T operating the vehicle V, etc.

Herein, the subject sensing data may be any biological, chemical, or physical data of the subject T that can be measured by sensors, etc.

For example, an example of the subject sensing data includes body temperature and body temperature distribution of the subject T. An example of the subject sensing data includes blood-related and cardiovascular system-related data like blood pressure value, heart rate, pulse wave, pulse wave velocity, electrocardiogram, arrhythmia state, blood flow rate, blood components such as blood glucose level. Examples of the blood components include red blood cell counts, white blood cell counts, platelet counts, pH value, electrolyte type, electrolyte quantity, hormone type, hormone quantity, and uric acid value, various markers, etc.

In addition, an example of the subject sensing data includes amount of perspiration, distribution of perspiration, skin resistance value, component of body odor, amount of digestive liquid such as saliva amount, components of digestive liquid such as saliva components. An example of the subject sensing data includes data on brain such as electroencephalogram, brain blood flow distribution, etc. An example of the subject sensing data includes data on respiration such as respiratory rate, respiratory volume, expiratory components, etc.

An example of the subject sensing data includes data on eyes such as number of blinks, amount of tears, eye movement (eye position, pupil diameter, etc.), etc. An example of the subject sensing data includes myoelectric data of each part of the body. An example of the subject sensing data includes data of facial color, facial expressions, etc.

An example of the subject sensing data includes data on sleeping such as bedtime, wakeup time, sleep duration, sleep pattern, snoring or not, strength of snoring, number of snoring, time of snoring, state of breathing, number of turns, posture during sleep, sleep quality like sleeping depth, etc. The sleep quality may be determined from, for example, the electroencephalogram, the eye movement, the breath, the posture during sleep, etc.

An example of the subject sensing data includes weight and height, etc. In addition, an example of the subject sensing data may include numerical data of symptoms of pain, numbness, etc.

In addition, an example of the measurement time point includes a time point when the measurement was started, a time point when the measurement was completed, or an intermediate time point between them to obtain a single value of the subject sensing data. The measurement time point may be a time point corresponding to measurement of a certain value. For example, in case that heart rate is calculated every minute, it may be any time point in this one minute. In addition, examples of such a time point include the peak time point of the R wave, the time point of the Q wave or the S wave, the peak time point of the P wave, etc., in case of calculating the heart rate from the length of time between the R waves in the electrocardiogram. Instead of time between R waves, it may be time between P waves, Q waves, S waves, or T waves, etc. In addition, not only in the electrocardiogram but also in graph of pulse waves, it may be a time point at which common characteristic points appear or an intermediate value as well. In case that blood pressure is measured by the Korotkoff sound, the measurement time point may be any time point within the measurement period when calculating the maximal blood pressure and the minimal blood pressure.

These subject sensing data above can be said to be the physiological data of the subject T. Incidentally, the data of seat pressure distribution may or may not be included in the physiological data.

Next, the disease assessment DB 12f stores the data necessary for the assessment of a predetermined disease. For example, as shown in FIG. 8, the disease assessment DB 12f stores the data necessary for the assessment of a predetermined disease in association with the disease ID, which indicates the type of disease, and the level of the disease.

The data necessary for the assessment of a predetermined disease includes, for example, statistically calculated operation values, movement values, the value of the relationship between the subject sensing data and the driving-characteristic data, the time that the gazing point is out of the center part of the subject T's visual field in the traveling direction of the vehicle V, and the value of the size of the seat pressure distribution, etc., from the data of multiple subjects with the same disease ID and disease level.

The data necessary for the assessment of a predetermined disease includes, for example, the operation values, etc., as well as thresholds for these values. The data necessary for the assessment of a predetermined disease includes, for example, the frequency ranges and the given frequencies when frequency analysis is performed. The data necessary for the assessment of a predetermined disease may be, for example, statistics of these values.

Based on operation-quantity data, movement-quantity data, or subject sensing data, spectral analysis, time series analysis, and other processes are performed, and statistics are calculated for multiple data. Examples of statistic include representative values such as average (arithmetic mean, geometric mean, harmonic mean, median, mode, maximum value, minimum value, etc.), dispersion, standard deviation, skewness, flatness, etc. The statistics may be calculated by performing multiple measurements on an individual subject. The value of the relationship, the operation values, the movement values, the time that the gazing point is out of the center part of the subject T's visual field in the traveling direction of the vehicle V, and the value of the size of the seat pressure distribution may be calculated for each classification of the road, from the operation-quantity data, movement-quantity data, or subject sensing data classified by the classification of the road.

Herein, the operation value is a value calculated from operation-related data which is data in the components of a predetermined frequency range, calculated from the operation-quantity data. The movement value is a value calculated from the movement-quantity data.

Operation-related data is data related to the operation-quantity data and calculated from the operation-quantity data. For example, examples of operation-related data include data and power spectrum density obtained by discrete Fourier transforming operation-quantity data, time-differentiated data obtained by time-differentiating the operation-quantity data, and time-integrated data obtained by time-integrating the operation-quantity data. One example of operation-related data in the components of a predetermined frequency range includes data in which the frequency components of a given frequency range are extracted from a spectrum or a power spectrum. The predetermined frequency range may be a single frequency or all frequencies within a range determined by sampling the data. The operation-related data in the components of the predetermined frequency range may be data by applying a filter, such as a low-pass filter, high-pass filter, or band-bass filter, to raw operation-quantity data. For example, it may be data with noise cut, data with predetermined frequency components emphasized, etc., to the raw operation-quantity data. The operation-related data in the components of the predetermined frequency range may be the Fourier transformed data of the operation-quantity data or the power spectrum itself. Incidentally, movement-related data is data related to movement-quantity data that is calculated from movement-quantity data. In addition, the subject sensing-related data is data related to the subject sensing data that is calculated from the subject sensing data. The same applies to the movement-related data and the subject sensing-related data regarding the above-mentioned operation-related data.

The value of the relationship between the subject sensing data and the driving-characteristic data may be, for example, the degree of dissociation between the gaze data and the operation-quantity data, the degree of dissociation between the gaze data and the movement quantity, and the value of the relationship may be the correlation coefficient between the subject sensing data and the driving-characteristic data.

The time that the gazing point is out of the center part of the subject T's visual field in the traveling direction of the vehicle V is the duration time that the gaze point is continuously (e.g., above 50 ms or 100 ms) out of the center part, the average time of the duration times, etc.

The value of the size of the seat pressure distribution is the seat pressure area, percentage, etc., in the case that the seat pressure is greater than a predetermined value.

The data in the disease assessment DB 12f may be data from when the subject T drives in a driving simulator or when he/she drives on a real road. Incidentally, for example, a standard highway driving simulator course has a total course length of 15.2 km, an average radius of curvature of 1640 m, and a height difference of 0.0 m. The driving simulator course on the Metropolitan Expressway in Tokyo has a total course length of 13.2 km, an average radius of curvature of 257 m, and a height difference of 17.5 m.

Herein, the data in the disease assessment DB 12f is explained using FIGS. 9 to 22, where the operation-quantity data, the movement-quantity data, and subject sensing data are measured for a healthy individual and a patient with epilepsy in case that the disease type is epilepsy.

Incidentally, examples of measurements when driving on a given course, where the classification of the road is Tokyo's Metropolitan Expressway, in a drive simulator are shown in FIGS. 9A to 21.

FIGS. 9A to 9C are graphs showing examples of gaze data and steering angle data when driving on a given course, where the classification of the road is Tokyo's Metropolitan Expressway, in a drive simulator. FIG. 9A shows data of a healthy subject. FIG. 9B shows data of a subject with epilepsy disease during non-seizure period. FIG. 9C shows data of a subject with epilepsy disease during seizure period.

The gaze data is shown by solid lines in the figure. Steering angle data is shown by dashed lines in the figure. The horizontal axis is time. In the case of the gaze data, the vertical axis (X-coordinate of gaze: X-AXIS OF GAZE POINT) indicates the value of gazing point in the horizontal direction relative to the traveling direction of the vehicle V, the upper direction of the vertical axis in the figure indicates the right direction of gaze, and the lower direction of the vertical axis in the figure indicates the left direction of gaze. In the case of steering angle data, the vertical axis (STEERING WHEEL ANGLE) is the angle of the steering angle, the upper direction of the vertical axis in the figure indicates clockwise and positive, and the lower direction of the vertical axis in the figure indicates counterclockwise and negative.

As shown in FIGS. 9A and 9B, the measurement results indicate that both the healthy individual and the patient with epilepsy disease show similar movements of gazing point and steering angle relative to time. The relatively slow (high-frequency component of the gaze data has been removed) lateral gaze movement is nearly identical to the movement of the steering angle. It was found that the high correlation between gaze data indicating lateral gaze and steering angle data. Incidentally, the spike-shaped movement of the gaze is a temporary movement of the gaze due to checking the rear view mirror, etc.

However, as shown in FIG. 9C, it was observed that, when the seizure started at time t0, the change in steering angle became weaker during seizure period (SEIZURE PERIOD), and the gaze deviated from the center part of the visual field rapidly and significantly. Incidentally, the seizures were identified by measuring electroencephalography.

The data necessary to determine epilepsy, such as the relationship between the gaze data and the steering angle data, is stored in the disease assessment DB 12f in association with an index ID for an indicator showing the relationship between gaze data and steering angle data, a sensor ID corresponding to the gaze data, an operation-quantity ID of the steering angle, a road classification ID indicating the road classification, and a disease ID indicating epilepsy, as the reference value. Incidentally, the threshold value of the relationship between the gaze data and the steering angle data may be set from the average values during non-seizure or seizure and stored in the disease assessment DB12f.

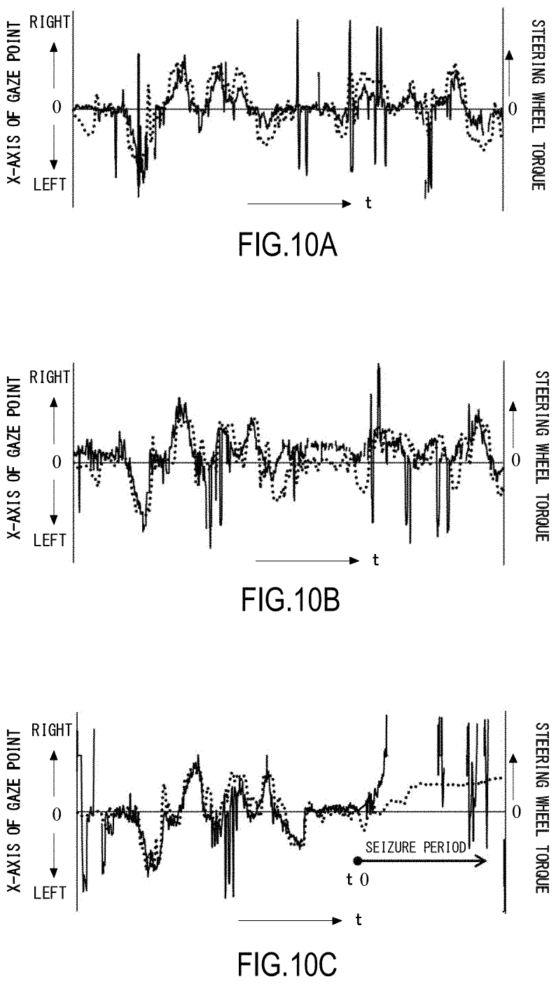

FIGS. 10A to 10C are graphs showing examples of gaze data and steering torque data when driving on a given course, where the classification of the road is Tokyo's Metropolitan Expressway, in a drive simulator. FIG. 10A shows data of a healthy subject. FIG. 10B shows data of a subject with epilepsy disease during non-seizure. FIG. 10C shows data of a subject with epilepsy disease during seizure.

The gaze data is shown by solid lines in the figure, as in FIG. 9. The steering torque data is shown by dashed lines in the figure. The horizontal axis indicates time. In the case of steering torque data, the vertical axis (STEERING WHEEL TORQUE) is the value of steering torque, the upper direction of vertical axis in the figure indicates clockwise and positive, and the lower direction of the vertical axis in the figure indicates counterclockwise and negative.

As shown in FIGS. 10A and 10B, the measurement results indicate that both the healthy individual and the patient with epilepsy disease show similar movements of gazing point and steering torque relative to time. The relatively slow (high-frequency component of the gaze data has been removed) lateral gaze movement is nearly identical to the movement of the steering torque. The high correlation between gaze data indicating lateral gaze and steering torque data was found.

However, as shown in FIG. 10C, when the seizure started at time t0, the change in steering torque became weaker during the seizure period, and the gaze was observed to rapidly and significantly deviate from the center part of the visual field.

The data necessary to determine epilepsy, such as the relationship between the gaze data and the steering torque data, is stored in the disease assessment DB 12f in association with an index ID for an indicator showing the relationship between gaze data and steering torque data, a sensor ID corresponding to the gaze data, an operation-quantity ID of the steering torque, a road classification ID indicating the road classification, and a disease ID indicating epilepsy, as the reference value. Incidentally, the threshold value of the relationship between the gaze data and the steering torque data may be set from the average values during non-seizure or seizure period and stored in the disease assessment DB12f.

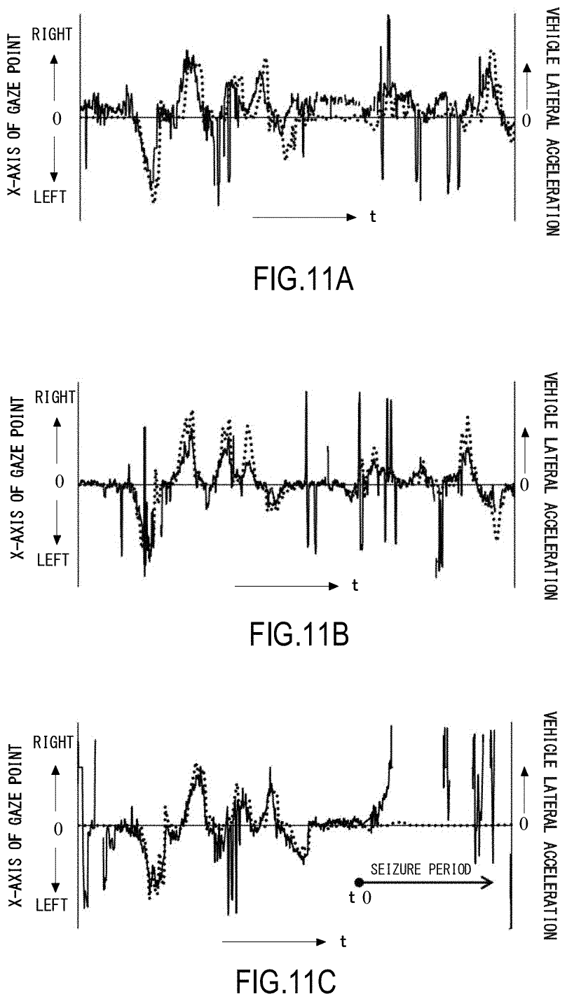

FIGS. 11A to 11C are graphs showing examples of gaze data and vehicle lateral acceleration data when driving on a given course, where the classification of the road is Tokyo's Metropolitan Expressway, in a drive simulator. FIG. 11A shows data of a healthy subject. FIG. 11B shows data of a subject with epilepsy disease during non-seizure period. FIG. 11C shows data of a subject with epilepsy disease during seizure.

The gaze data is shown by solid lines in the figure, as in FIG. 9. The vehicle lateral acceleration data is shown by dashed lines in the figure. The horizontal axis indicates time. In the case of vehicle lateral acceleration data, the vertical axis (vehicle lateral acceleration: VEHICLE LATERAL ACCELERATION) is the vehicle lateral acceleration of the vehicle V, the upper direction of vertical axis in the figure indicates the right direction with respect to the traveling direction of the vehicle V, and the lower direction of the vertical axis in the figure indicates the left direction with respect to the traveling direction of the vehicle V.

As shown in FIGS. 11A and 11B, the measurement results indicate that both the healthy individual and the patient with epilepsy disease show similar movements of gazing point and vehicle lateral acceleration relative to time. The relatively slow (high-frequency component of the gaze data has been removed) lateral gaze movement is nearly identical to the movement of the vehicle lateral acceleration. The high correlation between gaze data indicating lateral gaze and vehicle lateral acceleration data was found.

However, as shown in FIG. 11C, when the seizure started at time t0, the vehicle lateral acceleration became almost zero during seizure period, and the gaze was observed to rapidly and significantly deviate from the center part of the visual field.

The data necessary to determine epilepsy, such as the relationship between the gaze data and the vehicle lateral acceleration data, is stored in the disease assessment DB 12f in association with an index ID for an indicator showing the relationship between gaze data and vehicle lateral acceleration data, a sensor ID corresponding to the gaze data, a movement-quantity ID of the vehicle lateral acceleration, a road classification ID indicating the road classification, and a disease ID indicating epilepsy, as the reference value. Incidentally, the threshold value of the relationship between the gaze data and the vehicle lateral acceleration data may be set from the average values during non-seizure or seizure and stored in the disease assessment DB12f.

Next, FIG. 12A shows a comparison between a healthy individual and a subject with epilepsy disease before the onset of seizures and during seizure period in the graph of the elapsed time after the degree of dissociation exceeds a predetermined value, for the degree of dissociation (an example of the value of the relationship) indexed by the relationship between the gaze and the steering angle.

Herein, the vertical axis indicates the elapsed time (ELAPSED TIME). The horizontal axis indicates the dissociation (DEGREE OF DISSOCIATION). The degree of dissociation between the gaze and the steering angle is, for example, the difference between the two values calculated every 0.5 seconds after converting both the gazing point and the steering angle so that the maximum value is 100. The elapsed time is, for example, the time that has elapsed since the degree of dissociation exceeded D0th. In the figure, white circles indicate the degree of dissociation before the onset of seizures (non-seizure), black circles indicate the degree of dissociation at the onset of seizures, and crosses indicate the degree of dissociation for a healthy individual.

As shown in FIG. 12A, even during non-seizure, the degree of dissociation can be high for shorter elapsed time values. However, it was found that using both the degree of dissociation and the elapsed time since the dissociation can capture the characteristics during seizure period and that a clear distinction can be made between non-seizure and seizure times. As shown in FIG. 12A, for example, a threshold of the degree of dissociation Dth or higher and a threshold of elapsed time Tth or higher can sharply distinguish between seizure and non-seizure periods.

The data necessary to determine epilepsy, such as the degree of dissociation between the gaze data and the steering angle data, and the elapsed time of this degree of dissociation, is stored in the disease assessment DB 12f in association with an index ID for an indicator showing the gaze data and the steering angle data, and the elapsed time, a sensor ID corresponding to the gaze data, an operation-quantity ID of the steering angle, a road classification ID indicating the road classification, and a disease ID indicating epilepsy, as the reference value. Incidentally, the threshold value of the degree of dissociation between the gaze data and the steering angle data, and the degree of the elapsed time of this degree of dissociation may be set from the average values during non-seizure or seizure period and stored in the disease assessment DB12f.

Next, FIG. 12B shows a comparison between a healthy individual and a subject with epilepsy disease before the onset of seizures and at the onset of seizures in the graph of the elapsed time after the degree of dissociation exceeds a predetermined value, for the degree of dissociation (an example of the value of the relationship) indexed by the relationship between the gaze and the steering torque.

Herein, the degree of dissociation between the gaze and the steering torque is, for example, the difference between the two values calculated every 0.5 seconds after converting both the gazing point and the steering torque so that the maximum value is 100. The elapsed time is, for example, the time that has elapsed since the degree of dissociation exceeded D0th.

As shown in FIG. 12B, even during non-seizure period, the degree of dissociation can be high for shorter elapsed time values as in the case of the steering angle. However, it was found that both the degree of dissociation and the elapsed time since the dissociation can capture the characteristics during the seizure and that a clear distinction could be made between non-seizure and seizure times. As shown in FIG. 12B, for example, a threshold of the degree of dissociation Dth or higher and a threshold of elapsed time Tth or higher can sharply distinguish between seizure and non-seizure times.

The data necessary to determine epilepsy, such as the degree of dissociation between the gaze data and the steering torque data, and the elapsed time of this degree of dissociation, is stored in the disease assessment DB 12f in association with an index ID for an indicator showing the gaze data and the steering torque data, and the elapsed time, a sensor ID corresponding to the gaze data, an operation-quantity ID of the steering torque, a road classification ID indicating the road classification, and a disease ID indicating epilepsy, as the reference value. Incidentally, the threshold value of the degree of dissociation between the gaze data and the steering torque data, and the degree of the elapsed time of this degree of dissociation may be set from the average values during non-seizure or seizure and stored in the disease assessment DB12f.

Next, FIG. 12C shows a comparison between a healthy individual and a subject with epilepsy disease before the onset of seizures and at the onset of seizures in the graph of the elapsed time after the degree of dissociation exceeds a predetermined value, for the degree of dissociation (an example of the value of the relationship) indexed by the relationship between the gaze and the vehicle lateral acceleration.

Herein, the degree of dissociation between the gaze and the vehicle lateral acceleration is, for example, the difference between the two values calculated every 0.5 seconds after converting both the gazing point and the vehicle lateral acceleration so that the maximum value is 100. The elapsed time is, for example, the time that has elapsed since the degree of dissociation exceeded D0th.

As shown in FIG. 12C, even during non-seizure, the degree of dissociation can be high for shorter elapsed time values as in the case of the steering angle and the steering torque. However, it was found that both the degree of dissociation and the elapsed time since the dissociation can capture the characteristics during the seizure and that a clear distinction could be made between non-seizure and seizure times. As shown in FIG. 12C, for example, a threshold of the degree of dissociation Dth or higher and a threshold of elapsed time Tth or higher can sharply distinguish between seizure and non-seizure times.

The data necessary to determine epilepsy, such as the degree of dissociation between the gaze data and the vehicle lateral acceleration data and the elapsed time of this degree of dissociation, is stored in the disease assessment DB 12f in association with an index ID for an indicator showing the gaze data and the vehicle lateral acceleration data, and the elapsed time, a sensor ID corresponding to the gaze data, a movement-quantity ID of the vehicle lateral acceleration, a road classification ID indicating the road classification, and a disease ID indicating epilepsy, as the reference value. Incidentally, the threshold value of the degree of dissociation between the gaze data and the vehicle lateral acceleration data, and the degree of the elapsed time of this degree of dissociation may be set from the average values during non-seizure or seizure period and stored in the disease assessment DB12f.

As described above, it would be possible to distinguish between seizures and non-seizures in case of more than a predetermined degree of dissociation and more than a predetermined elapsed time, with respect to the degree of dissociation between the driving characteristics and the gaze of the subject T.

FIGS. 13A to 13C are graphs showing examples of the driving characteristics-related data obtained by frequency-analyzing the driving-characteristic data. The horizontal axis is the frequency and the vertical axis is the power spectral density (PSD) on a logarithmic scale. Herein, in the figure, symbol (a) shows the data from the start of the vehicle to before the start of the subject's seizure, symbol (b) shows the data in the section where the seizure occurred, symbol (c) shows the data during non-seizure period on the same subject, and symbol (d) shows the data on a healthy individual.

FIG. 13A is a graph showing an example of operation-related data obtained by Fourier transforming the steering angle data. As shown in FIG. 13A, the power spectral densities for both a subject with epileptic disease and a healthy subject were almost identical during non-seizure period (symbols (a), (c), and (d)). However, as shown in symbol (b), during seizure period, the power spectral density decreased over the entire frequency range.

FIG. 13B is a graph showing an example of operation-related data obtained by Fourier transforming the steering torque data. As shown in FIG. 13B, the power spectral densities for both a subject with epileptic disease and a healthy subject were almost identical during non-seizure period (symbols (a), (c), and (d)). However, as shown in symbol (b), during seizure period, the power spectral density decreased over the entire frequency range.

FIG. 13C is a graph showing an example of operation-related data obtained by Fourier transforming the vehicle lateral acceleration data. As shown in FIG. 13C, the power spectral densities for both a subject with epileptic disease and a healthy subject were almost identical during non-seizure period (symbols (a), (c), and (d)). However, as shown in symbol (b), during seizure period, the power spectral density decreased over the entire frequency range.

Thus, for example, as shown in FIG. 13A, the power spectral density p0 for frequency f0 (an example of operation values), the power spectral density p1 for frequency f1, etc. may be set as threshold values for assessment. In addition, as shown in FIG. 13B, the power spectral density p2 for frequency f2 (an example of operation values), etc. may be set as threshold values for assessment. As shown in FIG. 13C, the power spectral density p3 for frequency f3 (an example of movement values), etc. may be set as threshold values for assessment.

In addition, instead of a specific frequency, a frequency range may be set in advance. It may also be the difference between power spectral densities. It may be the square root of the integrated value of the difference in the power spectral densities in a specific frequency range, on the basis of the case of a healthy individual. Since it is sufficient to be able to distinguish disease conditions, the integral or sum of the power spectral densities may be used instead of calculating the square root.

The data necessary to determine epilepsy, such as these operation values or movement values, frequency ranges and frequency values, is stored in the disease assessment DB 12f in association with an operation-quantity ID or a movement-quantity ID, the road classification ID indicating the road classification, and the disease ID indicating epilepsy, as the reference operation value or movement value. Incidentally, specific power spectral density thresholds for a specific frequency may be set from average values during non-seizure and seizure periods and stored in the disease assessment DB 12f.

FIGS. 14A to 16B are graphs showing an example of duration time that the gaze point was continuously located outside the center part of the visual field. Herein, the visual field is, for example, the visual field of the subject T in the traveling direction of the vehicle V. The center part of the visual field shows range of the center part centered in front of the vehicle V when the subject T is seated in the seat of the vehicle V.

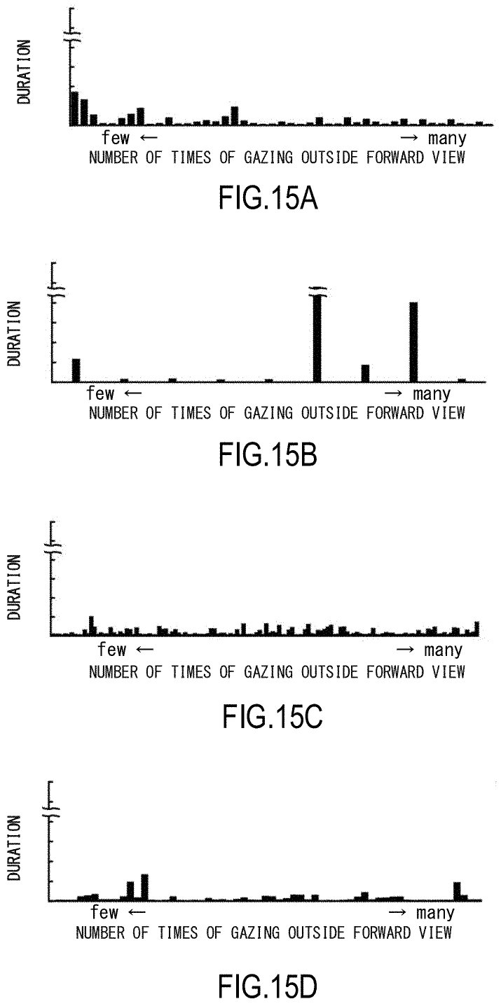

FIGS. 14A to 14D are cases where the range of the center part is set narrower. FIGS. 15A to 15D are cases where the range of the center part is set wider. Incidentally, in FIGS. 14A to 15D, the vertical axis (duration time: DURATION) is the duration time that the gaze point was continuously (e.g., above 50 ms or 100 ms) outside the center part. The horizontal axis (number of times the gaze point was positioned off-center: NUMBER OF TIMES OF GAZING OUTSIDE FORWARD VIEW) is an axis with the count in order in cases where the gaze point is consecutively outside the center part. That is, the horizontal axis is the total number of times the gaze point deviates from the set central range. The scale on this horizontal axis is indicated by “few” and “many”. FIGS. 14A and 15A show data from the start of the vehicle to before the start of the subject's seizure, FIGS. 14B and 15B show the data in the section where the seizure occurred, FIGS. 14C and 15C show the data during non-seizure period on the same subject, and FIGS. 14D and 15D show the data on a healthy individual.

Herein, the shape of the center part of the subject's visual field may be circular, oval, square, or rectangular, etc. The size of the center part of the subject's visual field is the diameter of a circle, the major and minor diameters of an oval, the length of one side or diagonal of a square, the length and width of a rectangle, the length of the diagonal of a rectangle, etc.

As shown in FIGS. 14A to 14D and FIGS. 15A to 15D, it can be seen that during seizure period, the gaze point is continuously located outside the center part for a long time. Herein, if the range of the center part is set small, the probability of detecting “gaze points continuously outside the center part” (sensitivity to epileptic seizure detection) increases, but if it is set too small, it may lead to false detection of an epileptic seizure. On the other hand, if the center part is set wider, the possibility of false detection is reduced, but the detection sensitivity is considered to be lower. Therefore, the setting of the center part should not be limited, and the epilepsy may be detected by varying the setting from small to large, or a combination of the two.

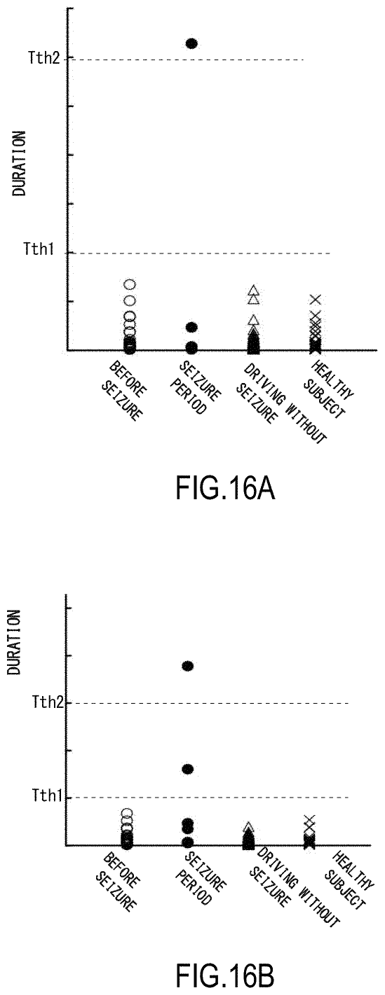

FIGS. 16A and 16B plot the respective duration times in the case from the start of the vehicle to before the start of the subject's seizure (BEFORE SEIZURE), in the section where the seizure occurred (SEIZURE PERIOD), during non-seizure period on the same subject (DRIVING WITHOUT SEIZURE), and on a healthy subject (HEALTHY SUBJECT). FIG. 16A is the case where the range of the center part of the visual field is set narrower. FIG. 16B shows the case where the range of the center part of the visual field is set wider.

As shown in FIG. 16A, in the case where the range of the center part of the visual field is set narrower, duration while the gaze point is continuously located outside the center part never exceeds Tth1 during non-seizure period, but it exceeds Tth2 during seizure period. As shown in FIG. 16B, in the case where the range of the center part of the visual field is set wider, Tth1 is never exceeded for duration while the gaze point is continuously located outside the center part during non-seizure period, but during seizure period, Tth2 is exceeded. Thus, as shown in FIGS. 16A and 16B, it is possible to distinguish the occurrence of epilepsy by thresholds Tth1 and Tth2 of the duration time.

The data necessary to determine epilepsy, such as these duration times, is stored in the disease assessment DB 12f in association with a sensor ID corresponding to the wearable terminal devices w2, the road classification ID, and the disease ID indicating epilepsy, as the reference subject sensing value. Incidentally, thresholds Tth1, Tth2, etc. of the duration time may be set from average values during non-seizure and seizure periods and stored in the disease assessment DB 12f.

FIGS. 17A and 17B show the average duration time (AVERAGE DURATION), in which the gaze point was continuously (e.g., above 50 ms or 100 ms) located outside the center part in the case from the start of the vehicle to before the start of the subject's seizure, in the section where the seizure occurred, during non-seizure period on the same subject, and on a healthy subject. FIG. 17A is the case where the range of the center part is set narrower. FIG. 17B shows the case where the range of the center part is set wider.

As shown in FIGS. 17A and 17B, the average duration time while the gaze point was continuously located outside the center part was found to be longer in seizure period. For example, as shown in FIGS. 17A and 17B, it is possible to distinguish the development of epilepsy by thresholds Tth1 and Tth2 of the average duration time.

The data necessary to determine epilepsy, such as these average duration time, is stored in the disease assessment DB 12f in association with a sensor ID corresponding to the wearable terminal devices w2, the road classification ID, and the disease ID indicating epilepsy, as the reference subject sensing value. Incidentally, threshold Tth, etc. of the duration time may be set from average values during non-seizure and seizure periods and stored in the disease assessment DB 12f.

FIGS. 18A and 18B show the rate (RATE OF DURATION), in which the gaze point was continuously (e.g., above 50 ms or 100 ms) located outside the center part in the case from the start of the vehicle to before the start of the subject's seizure, in the section where the seizure occurred, during non-seizure period on the same subject, and on a healthy subject. FIG. 18A is the case where the range of the center part is set narrower. FIG. 18B shows the case where the range of the center part is set wider.

Herein, the rate is calculated, for example, from “the number of frames in case outside the center part”/“total number of frames in a given period” in the video frames of the camera video capturing the eyes of subject T. Alternatively, the rate may be calculated from “time when the gaze point is located outside the center part”/“the total time in a given period.”

As shown in FIGS. 18A and 18B, the rate of duration while the gaze point is continuously located outside the center part never exceeds R1 during non-seizure period, but the rate of duration time during seizure period is approximately R2. Thus, as shown in FIGS. 18A and 18B, it is possible to distinguish the development of epilepsy by thresholds R1 and R2.

The data necessary to determine epilepsy, such as these rates, is stored in the disease assessment DB 12f in association with a sensor ID corresponding to the wearable terminal devices w2, the road classification ID, and the disease ID indicating epilepsy, as the reference subject sensing value. Incidentally, threshold R1, R2, etc. of the rate may be set from average values during non-seizure and seizure periods and stored in the disease assessment DB 12f.

Next, FIG. 19 is an example of the data of the seat pressure distribution measured by the sheet sensor ss2.

The degree of laterality (an example of subject sensing values) calculated from the data of the seat pressure distribution as shown in FIG. 19 is averaged over multiple measurements. The multiple measurements may be multiple measurements on the same person, or on multiple people with the same attributes. The data necessary for the determination of a predetermined disease, such as average of the degree of laterality, etc., is stored in the disease assessment DB 12f in association with the sensor ID corresponding to the sheet sensor ss, the road classification ID, and the disease ID, as the reference subject sensing value.

Herein, the degree of laterality is calculated from the center position of the seat pressure distribution. The center position is for example, the center of seat-pressure position COP (Center of Position) of the seat pressure distribution, which is calculated from the seat pressure distribution. The seat-pressure center of position is, for example, the center of gravity (Gx, Gy) of the seat pressure distribution. The center of gravity of the seat pressure distribution is a weighted average of the position of each point in the heat map of seat pressure with the value of the heat map at that position as the weight. In this case, the degree of laterality is the value Gx of the position of the center of gravity in the x-axis, starting from the origin (0, 0) of the sheet sensor ss. Incidentally, the seat-pressure center position may be a value calculated from the entirety of each point of the seat pressure heat map of the seat pressure distribution.

The center position may be, for example, the shape center position calculated from the shape of the seat pressure distribution. The shape center position is, for example, the COB (Center of Body). In this case, the degree of laterality is the value of the position of the COB in the x-axis, starting from the origin (0, 0) of the sheet sensor ss, which is an example of the center of left-right position on the vehicle V's seat. Incidentally, the COB is determined from the concave portion of the distribution shape. The front side, rear side, left side, and right side in the figure represent the direction from the subject T when he or she sits in the seat.

In addition, the degree of laterality may be the difference between the shape center position and the seat-pressure center position. For example, the degree of laterality is Gx-COB or COB-Gx.

FIGS. 20 and 21 are graphs showing an example of time series of seat-pressure center data and size of seat pressure distribution. In the figure, graph (a) is the x-coordinate position of the COP (COP COORDINATE), graph (b) is the y-coordinate position of the COP (COP COORDINATE), and graph (c) is the area of seat pressure (AREA OF SEAT PRESSURE), an example of the size of the seat pressure distribution. Herein, the area of seat pressure is indicated, for example, as the rate of the area where pressure is applied in the entire seating surface. In FIG. 19, the area of seat pressure is calculated as the rate of seat pressure above a predetermined value. FIG. 20 shows an example of data for a healthy subject. FIG. 21 shows an example of data for a subject with epilepsy disease at the time of epilepsy at t0 during a seizure.

As shown in FIG. 20, there was little change in the area of seat pressure even when the COP changed for the healthy individual. However, as shown in FIG. 21, the COP changed significantly at t0 during epileptic seizure and the area of seat pressure narrowed, that is, the area of the seat pressure was below the threshold value Sth.

The area of the seat pressure is averaged over multiple measurements for the data of the seat pressure distribution as well. The data necessary to determine epilepsy, such as the threshold for average of the area of the seat pressure, is stored in the disease assessment DB 12f in association with a sensor ID corresponding to the sheet sensor ss, each road classification ID, and each disease ID, as the reference value. Incidentally, the threshold value Sth of the area of the seat pressure may be set from the average values during non-seizure or seizure period and stored in the disease assessment DB12f.

The subject information DB 12a, the operation-quantity DB 12b, the movement-quantity DB 12c, driving-environment information DB 12d, the subject sensing DB 12e and disease assessment DB 12f may exist in the information processing server device 10, exist in another server connected to information processing server device 10 via a network, or be distributed in the network N. These may be separate databases, or be in the same database.

The output units 13 has, for example, liquid crystal display elements or electroluminescence (EL) devices in case of outputting image. The output units 13 has speakers in case of outputting sound.

The input unit 14 has, for example, a keyboard and a mouse.

The input/output interface unit 15 conducts interface processing between the communication unit 11, the memory unit 12, etc., and the control unit 16.

The control unit 16 has, for example a CPU (Central Processing Unit) 16a, a ROM (Read Only Memory) 16b, and a RAM (Random Access Memory) 16c. When the CPU 16a reads and executes various programs stored in the ROM 16b or the memory unit 12, the control unit 16 assesses disease condition of each subject T.

(2.2 Configuration and Functions of Mobile Terminal Device 20)

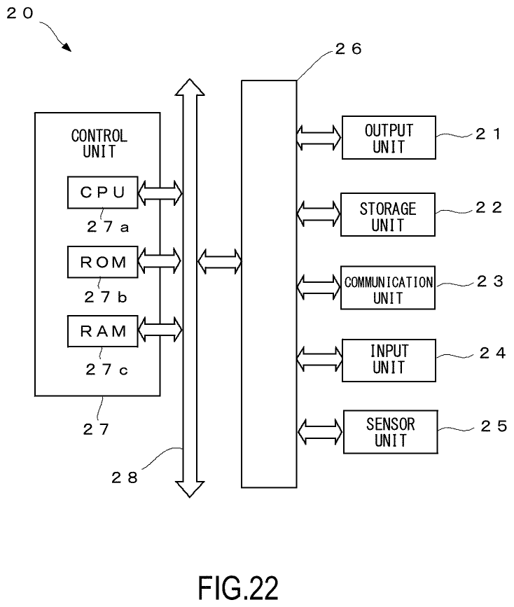

The following describes a configuration and functions of the mobile terminal device 20 using FIG. 22.

FIG. 22 is a block diagram showing an example of overview configuration of the mobile terminal device 20.

As shown in FIG. 22, the mobile terminal device 20 includes an output unit 21, a storage unit 22, a communication unit 23, an input unit 24, a sensor unit 25, an input/output interface unit 26 and a control unit 27. The control unit 27 and the input/output interface unit 26 are connected electrically via a system bus 28. In addition, a mobile terminal ID is assigned to each mobile terminal device 20. The mobile terminal device 20 has a clock function. The mobile terminal device 20 may have a vibration function for vibrating the mobile terminal device 20.

The output unit 21 has, for example, a liquid crystal display element or an EL element as a display function. The output unit 32 has a speaker that outputs sound.

The storage unit 22 includes, for example, hard disk drives or solid state drivers. The storage unit 22 stores various programs such as an operating system and apps for the mobile terminal device 20. Incidentally, the various programs may be obtained from, for example, another server device over the network N, or may be recorded in a recording medium and read via a drive device. In addition, the storage unit 22 may have the information of databases like the storage unit 12 of the information processing server device 10.

The communication unit 23 connects to the network N electrically or electromagnetically and controls the state of communications with, for example, the information processing server device 10. In addition, the communication unit 23 connects to the information processing server device 10 electrically or electromagnetically and controls the state of communications with, for example, the information processing server device 10.

The communication unit 23 has a function of wireless communication for conducting communication with a terminal device by radio waves or infrared rays. The mobile terminal device 20 communicates with the in-vehicle terminal device 30 and the home terminal device 40 via the communication unit 23. In addition, as shown in FIG. 2, the mobile terminal device 20 carried by the subject T communicates with the sheet sensor ss installed in the seat where the subject T is sitting and a wearable terminal devices w1, w2 worn by the subject T via the communication unit 23. Incidentally, the mobile terminal device 20 may conduct wired communication with the in-vehicle terminal device 30, the home terminal device 40, the sheet sensor ss and the wearable terminal devices w1, w2.

The communication unit 23 may communicate with IC tags as a leader of the IC tags.

The input unit 24 has, for example, a display panel of a touch switch type such as a touch panel. The input unit 24 acquires position information of the output unit 21 to which the user's finger touched or approached. The input unit 24 has a microphone for inputting voice sound.

The sensor unit 25 has various sensors such as a GPS (Global Positioning System) sensor, a direction sensor, an acceleration sensor, a gyro sensor, an atmospheric pressure sensor, a temperature sensor and a humidity sensor. The sensor unit 25 has imaging elements such as a CCD (Charge Coupled Device) image sensor and a CMOS (Complementary Metal Oxide Semiconductor) image sensor of a digital camera. The mobile terminal device 20 acquires current position information of the mobile terminal device 20 by the GPS sensor. Incidentally, a unique sensor ID is assigned to each sensor.

The input/output interface unit 26 conducts interface processing between the output unit 21 and the memory unit 22 etc., and the control unit 27.

The control unit 27 includes a CPU 27a, a ROM 27b, and a RAM 27c. In the control unit 27, the CPU 27a reads and executes various programs stored in the ROM 27b or the storage unit 22.

Herein, the wearable terminal device w1 is a wristband-type wearable computer. The wearable terminal device w1 has an output unit, a storage unit, a communication unit, an input unit, a sensor unit, an input/output interface unit, a control unit, and a timer unit (not shown).

The sensor unit of the wearable terminal device w1 measures various physiological data of the subject T.

The sensor unit has an acceleration sensor, a gyro sensor, a temperature sensor, a pressure sensor, an ultrasonic sensor, a light sensor, an electric sensor, a magnetic sensor, an image sensor, etc. Incidentally, a unique sensor ID is assigned to each sensor.

The acceleration sensor measures the acceleration of the wearable terminal device w1. From the measurement data of the acceleration sensor, the movement of the subject T's arm is measured. The gyro sensor measures the angular acceleration of the wearable terminal device w1. From the measurement data of the gyro sensor, the subject T's arm rotation is measured. The wearable terminal device w1 may measure the posture during sleeping, the number of turns, the number of steps, etc., by the acceleration sensor or the gyro sensor.

The temperature sensor measures the temperature of a contact parts or the temperature of parts imaged by the thermography. The pressure sensor measures, for example, pulse waves. The light sensor detects responses to irradiation of electromagnetic wave in the skin, etc., that is, at least one of a reflected wave and a transmitted wave. The light sensor measures the velocity of blood flow, blood components, etc.

The ultrasonic sensor detects responses to irradiated of ultrasonic waves, that is, at least one of a reflected wave and a transmitted wave.

The electric sensor measures voltage, current, impedance, etc. The electric sensor measures electric field generated by muscle work, blood flow, nerve excitation, etc. The electric sensor in combination with electrodes also detects components of sweat, etc., and functions as a chemical sensor, a pH sensor, etc.

The magnetic sensor measures the magnetic field generated by muscle work, blood flow, nerve excitation, etc.

The image sensor detects the color of skin, the surface temperature, the movement of the surface, the flow of blood flow, the appearance of sweat, etc.

In addition, the sensor unit has a GPS sensor, a direction sensor, an acceleration sensor, a gyro sensor, an atmospheric pressure sensor. The wearable terminal device w1 may measure the moving distance, the exercise amount, etc., by these sensors.

In addition, the microphone of the input unit may capture snoring and breath sound during sleeping in the subject T.

The subject sensing data measured by the sensor part of the wearable terminal device W1 or a sensor embedded in the contact parts with the subject, such as a steering wheel, is transmitted to the mobile terminal device 20 via the communication unit. Incidentally, the wearable terminal device W1 may transmit the measured subject sensing data to the in-vehicle terminal device 30.

Herein, the wearable terminal device w2, like the wearable terminal device w1 is an eyeglass-type wearable computer. The wearable terminal device w2 has an output unit, a storage unit, a communication unit, an input unit, a sensor unit, an input/output interface unit, a control unit, and a timer unit (not shown).

The sensor unit of the wearable terminal device w2 further has a sensor for measuring the movement of the gaze point. For example, in the case of the corneal reflection method, this sensor unit has an LED that emits light rays such as far infrared rays which is irradiated to the eyeball and a camera for eye tracking that captures images of the eyes of the subject T. The control unit of the wearable terminal device w2 calculates the reflection points on the cornea from the image and the subject T's gazing point from the position of the pupils, and outputs the gaze data from the output unit.

The wearable terminal device w2 mainly measures gazing point, blink, pupil size, etc.

Incidentally, the wearable terminal device w2 may specify the gazing point by distinguishing the white scleral area from the cornea by image processing from the image of the camera that captures the eyes of the subject T, without emitting far infrared rays or other rays from the LEDs in the sensor unit.

The sensor unit of the wearable terminal device w2 may be an eye tracker, with eye tracking functionality only. The wearable terminal device w2 may be a contact lens type. The sensor unit of the wearable terminal device w2 may also have a sensor that measures myoelectricity. This sensor unit may measure myoelectricity around the eye, calculate the direction of the eye, and obtain gaze data. The wearable terminal device w2 may also measure pulse rate, blood pressure, body temperature, etc. from the temple area.

Incidentally, as a type of the wearable terminal devices w1, w2, it may be a glasses type, a finger ring type, a shoe type, an in-pocket type, a necklace type, a garment type, etc., as well as the eyeglass type and the wristband type in FIG. 2.

(2.3 Configuration and Functions of In-Vehicle Terminal Device 30)

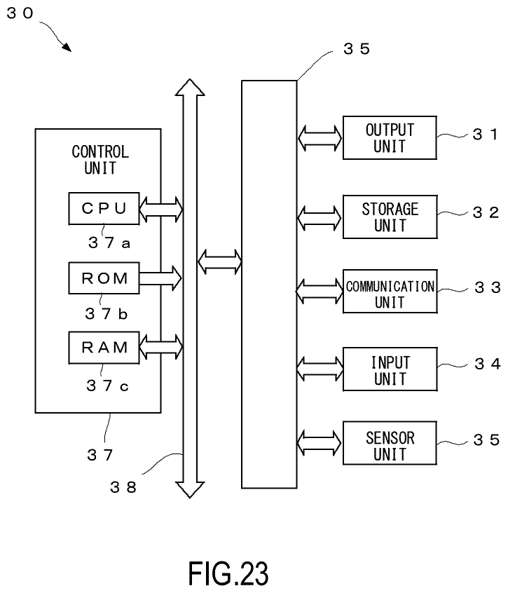

The following describes a configuration and functions of the in-vehicle terminal device 30 using FIG. 23.

FIG. 23 is a block diagram showing an example of overview configuration of the in-vehicle terminal device 30.

As shown in FIG. 23, the in-vehicle terminal device 30 includes an output unit 31, a storage unit 32, a communication unit 33, an input unit 34, a sensor unit 35, an input/output interface unit 36, and a control unit 37. The control unit 37 and the input/output interface unit 36 are connected electrically via a system bus 38. In addition, a vehicle ID is assigned to each in-vehicle terminal device 30. The in-vehicle terminal device 30 has a clock function.

As shown in FIG. 2, the in-vehicle terminal device 30 is, for example, a navigation device mounted on the vehicle V.

The output unit 31 has, for example, a liquid crystal display element or an EL element as a display function, and a speaker that outputs sound.

The storage unit 32 includes, for example, hard disk drives or solid state drivers. The storage unit 32 stores various programs such as an operating system and apps for the in-vehicle terminal device 30. Incidentally, the various programs may be obtained from, for example, another server device over the network N, or may be recorded in a recording medium and read via a drive device. In addition, the storage unit 32 may include the information of a database, such as the storage unit 12 of the information processing server device 10.

The storage unit 32 has map information for navigating the vehicle V.

Incidentally, in the storage unit 32, the subject information DB, the operation-quantity DB, the movement-quantity DB, the driving-environment information DB, and the subject sensing DB may be constructed for subject(s) T driving the vehicle V in which the in-vehicle terminal device 30 is installed, as in the storage unit 12.

The communication unit 33 connects to the network N electrically or electromagnetically and controls the state of communications with, for example, the information processing server device 10. In addition, the communication unit 33 connects to the information processing server device 10 electrically or electromagnetically and controls the state of communications with, for example, the information processing server device 10. The communication unit 33 controls communication with the mobile terminal device 20 by wireless communication. The communication unit 33 may communicate with the sheet sensor ss and the wearable terminal devices w1, w2.

The communication unit 33 communicates with the drive mechanism of the vehicle V. For example, a control signal is transmitted to the drive mechanism of the vehicle V via the communication unit 33 of the in-vehicle terminal device 30, and the vehicle V is stopped, stopped at a predetermined place, and navigated to a predetermined place such as a hospital.

The input unit 34 has, for example, a display panel of a touch switch type such as a touch panel. The input unit 34 acquires position information of the output unit 31 to which the user's finger touched or approached. The input unit 34 has a microphone for inputting sound.

The sensor unit 35 has various sensors to measure the operation quantity, such as an angle sensor for measuring the steering angle in the steering wheel sw, an accelerator pedal application sensor to measure the operation of the accelerator pedal, and a brake pedal application sensor to measure the operation of the brake pedal.

The sensor unit 35 has various sensors that measure the movement quantity of the vehicle V, such as a GPS sensor, a direction sensor, an acceleration sensor, a gyro sensor, and a sensor for millimeter wave radar. The GPS sensor acquires the current position information of the vehicle V.

The sensor unit 35 has various sensors such as an atmospheric pressure sensor, a temperature sensor, and a humidity sensor.

The sensor unit 35 has imaging elements such as a CCD image sensor and a CMOS image sensor of a digital camera. As shown in FIG. 2, the sensor unit 35 has a camera 35a and a camera 35b.

The camera 35a photographs circumstances outside the vehicle V. In the in-vehicle terminal device 30, the movement quantity of the vehicle V is measured by the images from the camera 35a. For example, fluctuation data, which is an example of movement-quantity data of the vehicle V, may be measured from images of lanes and scenery based on image data from cameras that capture the front, side, or rear of the vehicle V. The camera in the sensor unit 35 may measure the inter-vehicular distance, stop positions, and lane departure. The camera of the sensor unit 35 may measure the condition of the road surface (presence or absence of rain, presence or absence of snow, presence or absence of pavement, etc.), and measures the presence or absence of a human.

The camera 35b photographs subject T. From the image of the camera 35b, the in-vehicle terminal device 30 authenticates the subject T with facial recognition, measures the facial color of the subject T, and determines whether the subject T is dozing. In addition, the operation quantity may be measured from the movement of the subject T based on the image data of the camera that captures the interior of the vehicle V.

In addition, the camera 35b may be a camera for eye tracking. In this case, the light beam such as far infrared rays may be applied to the eyes and the camera 35b may capture the reflected light.

The input/output interface unit 36 conducts interface processing between the output unit 31 and the memory unit 32 etc., and the control unit 37.

The control unit 37 includes a CPU 37a, a ROM 37b and a RAM 37c. In the control unit 37, the CPU 37a reads and executes various programs stored in the ROM 37b or the storage unit 32.

[3. Operation Example of Disease-Condition Assessment System S]

The following describes an operation example of disease-condition assessment system S using figures.

(3.1 Collecting Data)

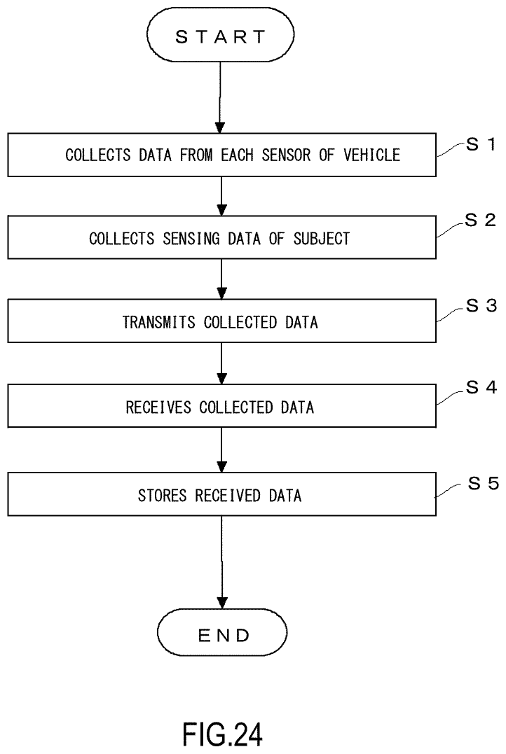

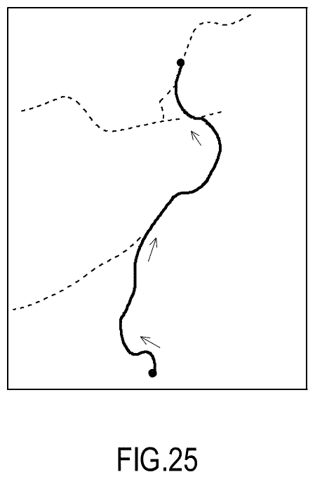

First, an operation example of collecting data, such as operation-quantity data, movement-quantity data, and subject sensing data of the subject T, is described using the figures. FIG. 24 is a flowchart showing an operation example of data collection. FIG. 25 is a schematic diagram showing an example of a road on which the vehicle traveled.

As shown in FIG. 2, the subject T gets in the vehicle V, and the power supply to the in-vehicle terminal device 30 is turned on. The in-vehicle terminal device 30 identifies the driver of the vehicle V. For example, the camera 35b of the in-vehicle terminal device 30 may photograph the subject T and conduct face recognition. The in-vehicle terminal device 30 may communicate with the mobile terminal device 20 or the wearable terminal devices w1, w2 of the subject T to identify the driver. The in-vehicle terminal device 30 may identify the driver with the fingerprint recognition sensor on the steering wheel of the vehicle V. The in-vehicle terminal device 30 may identify the driver by combining these driver identification methods. The subject T may be identified by the mobile terminal ID of the mobile terminal device 20 carried by the subject T.

Incidentally, the subject T may drive in a real vehicle or in a drive simulator.

When the subject T drives the vehicle V, the in-vehicle terminal device 30 starts measuring the operation-quantity data and the movement-quantity data. The mobile terminal device 20 measures sensing data.

Next, as shown in FIG. 24, the disease-condition assessment system S collects data from each sensor of the vehicle V (Step S1). Specifically, the control unit 37 of the in-vehicle terminal device 30 acquires the data measured by each sensor of the sensor unit 35 together with the measurement time point of the clock function, from each sensor. For example, the control unit 37 acquires the operation-quantity data such as steering angle data of the steering angle in the steering wheel sw, accelerator pedal application data, brake pedal application data, etc., from each sensor of sensor unit 35 as the operation quantity of vehicle V. In addition, the control unit 37 acquires fluctuation data, current position information of the vehicle V, the traveling direction of the vehicle V, the velocity, the acceleration, the inter-vehicular distance, etc., from each sensor of sensor unit 35 as the movement quantity of vehicle V.

The control unit 37 may acquire the image outside the vehicle V by the camera 35a of the sensor unit 35 as the fluctuation data. The control unit 37 acquires the image of the subject T by the camera 35b. Incidentally, the measurement time point measured by each sensor of the sensor unit 35 may be measured by the clock function of the in-vehicle terminal device 30.

Next, the disease-condition assessment system S collects the sensing data of the subject T from the sensors of the wearable terminal device w1, w2 and the sheet sensor ss (Step S2). Specifically, the control unit 27 of the mobile terminal device 20 of the subject T who is driving the vehicle V acquires the sensing data measured by each of the sensors of the wearable terminal device w1, w2 and the sheet sensor ss using the wearable terminal device w1, w2. The control unit 27 acquires the gaze data of the subject T driving the vehicle V from the wearable terminal device w2. The control unit 27 acquires the rotational data of the arm rotation of the subject T who is operating the vehicle V from the wearable terminal devices w1 on both arms. The control unit 27 acquires the data of the seat pressure distribution on the seating surface where the subject T operating the vehicle V sits from the sheet sensor ss.

The measurement time may be measured by a clock function of the mobile terminal device 20 or by a clock function of the wearable terminal device w1, w2.

Next, the in-vehicle terminal device 30 acquires the sensing data via the mobile terminal device 20. Incidentally, the mobile terminal device 20 may acquire the operation-quantity data and the movement-quantity data via the in-vehicle terminal device 30.

The measured data may be stored in each terminal device. When storing the measured data in the storage unit 32, the in-vehicle terminal device 30 may store the measured data in the storage unit 32 in association with the subject ID, the operation-quantity ID, the movement-quantity ID, and the sensor ID. Alternatively, the mobile terminal device 20 may store the measured data in the storage unit 22 in association with the subject ID, the operation-quantity ID, the movement-quantity ID, and the sensor ID.

Next, the disease-condition assessment system S transmits the collected data to the information processing server device 10 (Step S3). Specifically, the in-vehicle terminal device 30 transmits the acquired data to the information processing server device 10. More specifically, the control unit 37 transmits the operation-quantity data of the vehicle V, the measurement time, the measurement position, the subject ID and the operation-quantity ID, to the information processing server device 10. The control unit 37 transmits the movement-quantity data of the vehicle V, the measurement time, the measurement position, the subject ID, and the movement-quantity ID, to the information processing server device 10. The control unit 37 transmits the gaze data of the subject T, the measurement time, the measurement position, the subject ID, and the sensor ID of the sensor for measuring the gaze movement to the information processing server device 10. The control unit 37 transmits the data of the seat pressure distribution of the subject T, the measurement time, the measurement position, the subject ID, and the sensor ID of the data of the seat pressure distribution, to the information processing server device 10.

The control unit 37 may transmit the vehicle ID instead of the subject ID. The mobile terminal device 20 may transmit the sensing data to the information processing server device 10. The mobile terminal device 20 may transmit the operation-quantity data and movement-quantity data.

The measured data may be transmitted to the information processing server device 10 sequentially or collectively. In the case of sequential transmission, the in-vehicle terminal device 30 may transmit a predetermined amount of data in packets, or the data may be transmitted in batches when communication is interrupted due to poor communication conditions, such as in a tunnel.

In addition, when transmitting the data collectively, the in-vehicle terminal device 30 may transmit predetermined data such as measured data in a predetermined driving section, measured data in a predetermined driving period, etc. Alternatively, the in-vehicle terminal device 30 may transmit the measured data collectively to the information processing server device 10 after driving is completed.

Next, the disease-condition assessment system S receives the collected data from the in-vehicle terminal device 30 (Step S4). Specifically, the information processing server device 10 receives the operation-quantity data of the vehicle V and the movement-quantity data of the vehicle V from the in-vehicle terminal device 30. The information processing server device 10 receives sensing data, such as gaze data and data of seat pressure distribution, from the in-vehicle terminal device 30.

In this manner, the information processing server device 10 functions as an example of the driving-characteristic data acquiring means for acquiring driving-characteristic data indicating driving characteristics of the subject for the vehicle. The information processing server device 10 functions as an example of the gaze data acquiring means for acquiring gaze data indicating a subject's gazing point measured when the subject is driving a vehicle. The information processing server device 10 functions as an example of the seat pressure distribution acquiring means for acquiring data of a seat pressure distribution on a seating surface where the subject is seated in the vehicle.

Next, the information processing server device 10 stores the received data in the storage unit 12 (Step S5). Specifically, the control unit 16 of the information processing server device 10 stores the received operation-quantity data, measurement time, position information, etc., in the operation-quantity DB 12b in association with the subject ID and operation-quantity ID. The control unit 16 stores the received movement-quantity data, measurement time, position information, etc., in the movement-quantity DB 12c in association with the subject ID and the movement-quantity ID. The control unit 16 stores the received sensing data, measurement time, position information, etc., in the subject sensing DB 12e in association with the subject ID and sensor ID.

As shown in FIG. 25, the road on which the vehicle V traveled is specified from the received position information of the vehicle V. Incidentally, the road to be traveled may be set in advance by the navigation function of the in-vehicle terminal device 30.

In addition, the information processing server device 10 acquires driving-environment information from the driving information provision server devices, and the data in the driving-environment information DB 12d is updated.

(3.2 Operation Example of Assessing Disease Condition)

Next, an operation example of assessing a disease condition for a particular subject T is explained using figures.

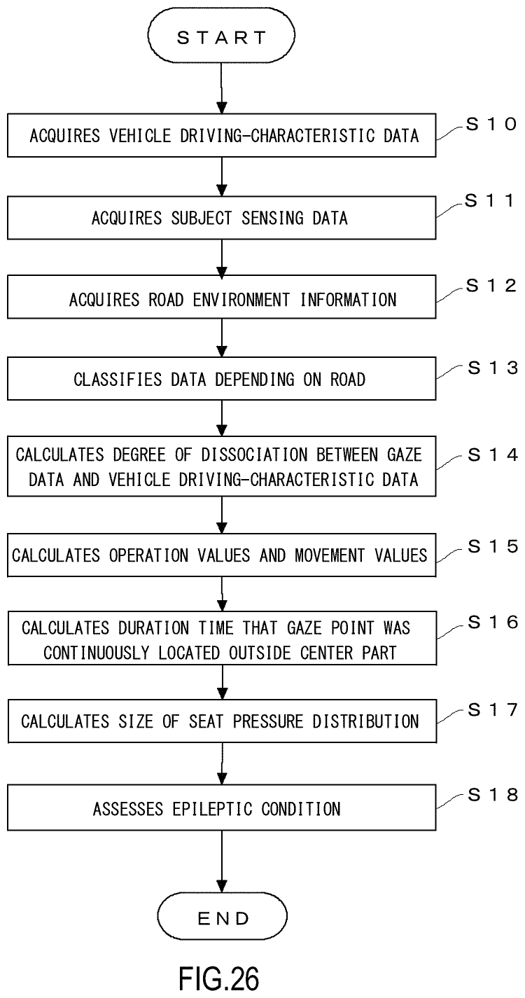

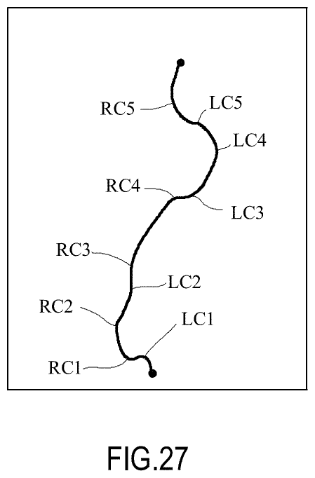

FIG. 26 is a flowchart showing an operation example of assessing the disease condition, such as epilepsy. FIG. 27 is a schematic diagram showing an example of the road on which the vehicle V traveled.

As shown in FIG. 26, the information processing server device 10 acquires the vehicle driving-characteristic data, such as operation-quantity data of the subject T operating the vehicle V and movement-quantity data of movement of the vehicle (Step S10).

In case of the operation-quantity data, the control unit 16 of the information processing server device 10 acquires each operation-quantity data such as steering angle data and accelerator pedal application data, measurement time, and position information of the vehicle V based on the subject ID of the subject T and each operation-quantity ID, with reference to the operation-quantity DB 12b. For example, the control unit 16 acquires each operation-quantity data when driving on the road as shown in FIG. 25.

In case of the movement-quantity data, the control unit 16 acquires each movement-quantity data such as fluctuation data, vehicle velocity data, lateral acceleration data, etc., measurement time, and position information of the vehicle V based on the subject ID of the subject T and each movement-quantity ID with reference to the movement-quantity DB 12c. For example, the control unit 16 acquires each movement-quantity data when driving on the road as shown in FIG. 25.

The information processing server device 10 functions as an example of the driving-characteristic data acquiring means for acquiring driving-characteristic data indicating driving characteristics of the subject for the vehicle.

Next, the information processing server device 10 acquires the subject sensing data (Step S11). Specifically, the control unit 16 acquires each subject sensing data, such as gaze data, data of seat pressure distribution, etc., measurement time, and position information of the vehicle V based on the subject ID of the subject T and each sensor ID in the subject sensing DB 12e. For example, the control unit 16 acquires the sensing data of each subject when driving on the road as shown in FIG. 25.

In this manner, the information processing server device 10 functions as an example of the gaze data acquiring means for acquiring gaze data indicating a subject's gazing point measured when the subject is driving a vehicle. The information processing server device 10 functions as an example of the seat pressure distribution acquiring means for acquiring data of a seat pressure distribution on a seating surface where the subject is seated in the vehicle.

Next, the information processing server device 10 acquires the road environment information (Step S12). Specifically, the control unit 16 acquires the road environment information of the traveled road as shown in FIG. 25 with reference to the driving-environment information DB 12d.

Next, the information processing server device 10 classifies the data depending on the road (Step S13). Specifically, the control unit 16 classifies the vehicle driving-characteristic data, such as each operation-quantity data and each movement-quantity data, based on the position information of the vehicle V at the time when these data were measured and the acquired road environment information. More specifically, the control unit 16 divides these data into each operation-quantity data and each movement-quantity data for position traveling on the standard highway, and each operation-quantity data and each movement-quantity data for position traveling on a highway with relatively many curves, like the Metropolitan Expressway in Tokyo. As described above, the control unit 16 classifies the data according to the type of the road on which the vehicle V is traveling.

The control unit 16 may calculate the curvature of the road from each position information and classify curves depending on the curvature to sort out to which section each operation-quantity data and each movement-quantity data belong. The control unit 16 may set roads with a predetermined curvature or less as straight and others as curved sections, and classify each operation-quantity data and each movement-quantity data depending on straight and curved sections. As shown in FIG. 27, curves may be classified to left curves and right curves. As shown in FIG. 27, curves may be classified for each curve above a predetermined curvature.

In this manner, the information processing server device 10 classifies operation-quantity data depending on the degree of curve of the road on which the vehicle is traveling.

Next, the control unit 16 specifies the road classification ID for each classified data from the position information with reference to the driving-environment information DB 12d. Incidentally, from the curvature or position information, the control unit 16 may specify the road classification IDs of roads that have similar road curvature patterns.

For each subject sensing data, such as gaze data, data of seat pressure distribution, etc., as well as each operation-quantity data and each movement-quantity data, it is sorted depending on the road and the road classification ID is identified.

Next, the information processing server device 10 calculates the degree of dissociation between the gaze data and the vehicle driving-characteristic data (Step S14). Specifically, the control unit 16 normalizes the acquired time-series gaze data and the vehicle driving-characteristic data so that the minimum value is 0 and the maximum value is 100. For example, the control unit 16 normalizes the gaze data of lateral gaze movement, steering angle data, steering torque data, and vehicle lateral acceleration so that the minimum value is 0 and the maximum value is 100.

The control unit 16 calculates the difference value (e.g., the absolute value of the difference) between the normalized gaze data and the normalized vehicle driving-characteristic data. The control unit 16 calculates the sum or average, etc., of the difference values over a given time length as the degree of dissociation. For example, the control unit 16 calculates the degree of dissociation between gaze and steering angle from the difference value between the gaze data of the normalized lateral gaze movement and the steering angle data. The control unit 16 calculates the degree of dissociation between gaze and steering torque from the difference value between the gaze data of the normalized lateral gaze movement and the steering torque. The control unit 16 calculates the degree of dissociation between gaze and vehicle lateral acceleration from the difference value between the gaze data of the normalized lateral gaze movement and the vehicle lateral acceleration data.

Incidentally, the control unit 16 may calculate a moving average or exponential smoothing of the time series of difference values of the normalized gaze data and vehicle driving characteristic data.

Next, the control unit 16 calculates each elapsed time since each degree of dissociation exceeds the threshold value D0th, from the time series of each degree of dissociation. For example, if a certain degree of dissociation is set (e.g., 20 or 30 when the normalized maximum value is 100) and the set degree of dissociation is exceeded, the control unit 16 measures the elapsed time from the time at which the set degree of dissociation was exceeded.

Next, the information processing server device 10 calculates, from the acquired gaze data, the duration time or average time that the gazing point is continuously (e.g., for 100 ms or more) located outside the set center part of the visual field. The information processing server device 10 calculates, from the acquired gaze data, the rate in which gazing point that was continuously located outside the center part of the set visual field.

The information processing server device 10 may calculate the duration time and average time of being located in the set center part, the rate of being located outside the center part, etc., using only the gaze data of lateral gaze movement.

For example, with regard to the duration time, the information processing server device 10 calculates, as the duration time, the elapsed time during which the gaze point has been continuously positioned outside the set central part beyond the set continuous time, in the case where the range of the center part of the visual field is set within the forward frontal view from sitting on the driver's seat and where a time (e.g., 100 ms or more) is set for the gaze point to be continuously outside the set center part.

With regard to the average time, in some case, the gaze point is positioned outside the center part for more than a set continuous time, then returns to within the center part, and then repeats the condition of being positioned outside the center part again. In this case, the information processing server device 10 calculates, as the “average time”, the average of multiple “duration times of gaze points consecutively located outside the center part” in each period (e.g., seizure period).

With regard to the rate, the information processing server device 10 calculates the rate of time (number of frames) that the gaze point was continuously positioned outside the center part to the total time (number of frames) of each period (e.g., seizure period).

Next, the information processing server device 10 calculates the operation-related data. Specifically, the control unit 16 calculates the operation-related data in the components of a predetermined frequency range from the operation-quantity data. For example, the control unit 16 calculates the power spectral density of each frequency by performing a discrete Fourier transform of the operation-quantity data.

Next, the control unit 16 specifies the predetermined frequency range based on the road classification ID, disease ID, and operation-quantity ID with reference to the disease determination DB 12f. The control unit 16 extracts the components of the power spectrum corresponding to the portion of the components in a predetermined frequency range from the power spectrum density as operation-related data.

In case that the operation-quantity data is steering angle data, the operation-related data may be the steering angular velocity (steering torque) calculated from the time-differentiating of the steering angle data.

Incidentally, the control unit 16 may calculate the movement-related data in the components of a predetermined frequency value or a predetermined frequency range from the movement-quantity data. For example, the control unit 16 may perform a discrete Fourier transform of the movement-quantity data and calculate the power spectral density of each frequency as movement-related data.

Next, the information processing server device 10 calculates operation values and movement values (Step S15). Specifically, the control unit 16 calculates time-series operation-related data and frequency analysis values that quantify the spectrum of operation-related data, which is a function of frequency, with reference to the disease determination DB 12f based on the road classification ID, disease ID, and operation-quantity ID. Based on the road classification ID, disease ID, and operation-quantity ID, the control unit 16 calculates the time-series movement-quantity data and the frequency analysis value that quantifies the spectrum of the movement-quantity data, which is a function of frequency, with reference to the disease determination DB 12f.

For example, in case that the disease ID indicates epilepsy, the road classification ID indicates Tokyo's Metropolitan Expressway, and the operation-quantity ID indicates steering angle, the control unit 16 calculates the power spectral density at the frequency values f0, f1 as shown in FIG. 13A or a combination of these frequencies. In case that the disease ID indicates epilepsy, the road classification ID indicates Tokyo's Metropolitan Expressway, and the operation-quantity ID indicates steering torque, the control unit 16 calculates the power spectral density at the frequency value f2 as shown in FIG. 13. In case that it is not a predetermined frequency value, but a predetermined frequency range, the control unit 16 calculates the total value, which is the sum of the power spectral densities in a predetermined frequency range, as the frequency analysis value.

Incidentally, the operation value may be the total steering quantity, which is calculated by integrating the absolute value of the steering angular velocity. The operation value may be the corrective steering quantity, which is the sum of the power spectral densities in a predetermined frequency band, obtained by discrete Fourier transforming the steering angle data. The operation values may be standard deviation of steering angular velocity, steering smoothness, maximum value of steering angular velocity and steering entropy which is the calculated entropy of steering angle data.

In addition, as a movement value, the control unit 16 calculates the degree of fluctuation (e.g., SDLP) when driving on the road indicated by the road classification ID from the fluctuation data. Incidentally, the degree of fluctuation on the vehicle V may be the number or frequency of departures from the road (frequency of sending lane departure warnings). The movement-quantity data as the inter-vehicular distance may be the value or variation degree of the inter-vehicular distance (or inter-vehicular time), the number and frequency of approaches (e.g., the inter-vehicular time is within 3 seconds, within 1 second, etc.) to the vehicle traveling in front (frequency of sending a forward collision warning). The distance from the location of the pause when the car stops can be regarded as movement-quantity data. From these movement-quantity data, the degree of fluctuation may be calculated as a movement value.

As a movement value, the control unit 16 calculates the average vehicle velocity when driving on the road indicated by the road classification ID from the vehicle velocity data. As a movement value, the control unit 16 calculates the average lateral acceleration value when driving on the road indicated by the road classification ID from the lateral acceleration data.

Incidentally, the control unit 16 may calculate the movement values from the movement-related data. For example, the control unit 16 calculates the total value, which is the sum of the power spectral densities in a predetermined frequency range as the movement value.

Next, the information processing server device 10 calculates the duration time that the gaze point was continuously located outside the center part of the visual field (Step S16). Specifically, the control unit 16 determines whether or not the gaze is out of the predetermined range of the center part based on the gaze data. Incidentally, it may be determined by multiple predetermined ranges of the center part, as shown in FIGS. 16A and 16B, etc.

In case of being out of the range of the center part, the control unit 16 calculates the duration while the gaze is continuously out of the predetermined range of the center part. If the duration time is less than, for example, 100 ms, it is not counted and truncated.

The control unit 16 calculates the average value of the duration time, i.e., the average time, in a predetermined period for the gaze data. In addition, it calculates, during a predetermined period in the gaze data, the rate of duration time that the gaze point was continuously located outside the center part.

Next, the information processing server device 10 calculates a center position depending on the seat pressure distribution. In case that the center position is the shape center position, the control unit 16 scans from the maximum value of y, that is, the line in front of the subject T in order from the minimum value of x, in the distribution map of the seat pressure distribution as shown in FIG. 19. After scanning and reaching the outer edge of the distribution shape, the control unit 16 stores the value of y, along with the value of x, as the distance from the frontal line to the outer edge of the distribution shape. The control unit 16 increments the value of x and scans from the frontal line. The control unit 16 repeats scanning these up to the maximum value of x. After the scanning is completed, the control unit 16 calculates the x position where the distance from the frontal line to the outer edge of the distribution shape becomes maximal, as the shape center position of the distribution shape. As shown in FIG. 25, if the maximal value is two or more, the averaged position is the shape center position. Incidentally, the method of calculating the shape center position is not limited to the above method, but it is sufficient if the concave portion of the distribution shape that separates the left and right sides of the seat pressure distribution can be calculated.

The outer edge of the distribution shape is where the seat pressure value is equal to or higher than a predetermined value in the distribution map of the seat pressure distribution. Incidentally, the control unit 16 may calculate a plurality of x-axis directional positions for the concave portion of the distribution shape by varying the predetermined value, and use the averaged position as the shape center position.

In case that the center position is the seat-pressure center position, the control unit 16 calculates the distribution shape center of gravity (Gx, Gy) as the seat-pressure center position from the value and position of each pixel in the distribution map of the seat pressure distribution.

Next, the information processing server device 10 calculates a degree of laterality as the subject sensing value. Specifically, the control unit 16 calculates the difference between the shape center position and the seat-pressure center position.

Next, the information processing server device 10 calculates a size of the seat pressure distribution (step S17). Specifically, the control unit 16 counts pixels or unit compartments where the seat pressure is above a predetermined value, as shown in FIG. 19. The control unit 16 divides the count by the number of pixels of the entire seating surface or the number of unit compartments, and calculates the rate of the part where pressure is applied in the entire seating surface as the area of seat pressure.

In this manner, the information processing server device 10 functions as an example of the pressure distribution calculation means for calculating a size of the seat pressure distribution.

Next, the information processing server device 10 assesses disease condition, such as epilepsy (Step S18). Specifically, the control unit 16 compares the reference operation value with the calculated operation value of the subject T with reference to the disease assessment DB 12f based on the road classification ID, disease ID, and operation-quantity ID, and assesses disease condition, such as whether the subject T has the disease of the disease ID and the disease severity of the disease ID.

For example, in case that the disease ID indicates epilepsy, the road classification ID indicates Tokyo's Metropolitan Expressway, the sensor ID indicates gaze, and the operation-quantity ID indicates steering angle, the control unit 16 compares the degree of dissociation calculated in step S14 and the elapsed time since the degree of dissociation exceeded D0th with the threshold values Dth for the degree of dissociation and Tth for the elapsed time as shown in FIG. 12A, to assess whether or not the condition is epilepsy. If the threshold value of dissociation Dth and the threshold value of elapsed time Tth are exceeded, the control unit 16 assesses that it is in an epileptic condition.

In case that the disease ID indicates epilepsy, the road classification ID indicates Tokyo's Metropolitan Expressway, the sensor ID indicates gaze, and the operation-quantity ID indicates steering torque, the control unit 16 compares the degree of dissociation calculated in step S14 and the elapsed time since the degree of dissociation exceeded D0th with the threshold values Dth for the degree of dissociation and Tth for the elapsed time as shown in FIG. 12B, to assess whether or not the epileptic condition or not.

In case that the disease ID indicates epilepsy, the road classification ID indicates Tokyo's Metropolitan Expressway, the sensor ID indicates gaze, and the movement-quantity ID indicates vehicle lateral acceleration, the control unit 16 compares the degree of dissociation calculated in step S14 and the elapsed time since the degree of dissociation exceeded D0th with the threshold values Dth for the degree of dissociation and Tth for the elapsed time as shown in FIG. 12C, to assess whether or not the epileptic condition or not.

In this manner, the information processing server device 10 functions as an example of the disease-condition assessment means for being capable of assessing an epileptic condition of the subject depending on the relationship between the gaze data and the driving-characteristic data. The information processing server device 10 functions as an example of the disease-condition assessment means for assessing that the disease condition is epilepsy when the value of the relationship is below a predetermined value. The information processing server device 10 functions as an example of the disease-condition assessment means assesses that the disease condition is epilepsy, when a time for which the degree of dissociation is greater than or equal to a predetermined value is greater than or equal to a predetermined time.

The following describes the case of operation-related data obtained by Fourier transforming the data.

For example, in case that the disease ID indicates epilepsy, the road classification ID indicates Tokyo's Metropolitan Expressway, and the operation-quantity ID indicates steering angle, the control unit 16 compares the operation values of the subject T at the frequency values f0 and f1 as shown in FIG. 13A with the power spectral density p0 for frequency f0 and the power spectral density p1 for frequency f1, as calculated in step S15, to assess whether or not the epileptic condition or not. If each calculated operation value is lower than the power spectral densities p0 and p1, the control unit 16 assesses that it is in an epileptic condition.

In case that the disease ID indicates epilepsy, the road classification ID indicates Tokyo's Metropolitan Expressway, and the operation-quantity ID indicates steering torque, the control unit 16 compares the operation values of the subject T at the frequency value f2 as shown in FIG. 13B with the power spectral density p2 for frequency f2, as calculated in step S15, to assess whether or not the epileptic condition or not.

In case that the disease ID indicates epilepsy, the road classification ID indicates Tokyo's Metropolitan Expressway, and the movement-quantity ID indicates vehicle lateral acceleration, the control unit 16 compares the operation values of the subject T at the frequency value f3 as shown in FIG. 13C with the power spectral density p3 for frequency f3, as calculated in step S15, to assess whether or not the epileptic condition or not.

Incidentally, the information processing server device 10 may assess the disease condition based on a single operation value or movement value, or based on multiple operation values.

For example, in case that assessments based on multiple operation values or movement values, the information processing server device 10 may assess the predetermined disease condition if the number of assessments for a predetermined disease condition exceeds a predetermined threshold. The information processing server device 10 calculates the sum of the assessment results (number of assessments) for each operation value, assuming that the assessment result is 1 if it is a predetermined disease condition and that the assessment result is 0 if it is not a predetermined disease condition. In addition, the information processing server device 10 may set a weight for each operation value and calculate the sum of the assessment results.

In case of assessing a combination of operation values and movement values, the information processing server device 10 may assess the predetermined disease condition if the number of assessments for a predetermined disease condition exceeds a predetermined threshold in assessing each operation value and each movement value. In addition, the information processing server device 10 may set a weight for each operation value and each movement value, and calculate the sum of the assessment results.

In this manner, the information processing server device 10 functions as an example of the disease-condition assessment means for being capable of assessing an epileptic condition of the subject depending on the relationship between the gaze data and the driving-characteristic data.

The following describes the case of subject sensing data.

Based on the road classification ID, disease ID, and sensor ID, the control unit 16 compares the reference subject sensing value with the calculated subject sensing value of the subject T, with reference to the disease assessment DB 12f, and the control unit 16 assesses the disease condition, such as whether the subject T has the disease of the disease ID and the disease severity of the disease ID.

For example, the following specifically describes the case of assessing gaze data.

In case that the disease ID indicates epilepsy, the road classification ID indicates Tokyo's Metropolitan Expressway, the sensor ID indicates gaze data, the control unit 16 compares the duration time that are continuously out of the predetermined range of the center part, which is calculated in step S16 with the threshold values Tth1 and Tth2 as shown in FIGS. 16A and 16B, to assess whether or not the epileptic condition or not. If the calculated duration time exceeds the threshold Tth1, or exceeds the threshold Tth2, or a combination of these cases, the control unit 16 assesses that it is in an epileptic condition.

In case that the disease ID indicates epilepsy, the road classification ID indicates Tokyo's Metropolitan Expressway, the sensor ID indicates gaze data, the control unit 16 compares the average value of the duration time that are continuously out of the predetermined range of the center part calculated in step S16 with the threshold value Tth as shown in FIGS. 17A and 17B, to assess whether or not the epileptic condition or not. If the calculated average value of duration time exceeds the threshold Tth, the control unit 16 assesses that it is in an epileptic condition.

In case that the disease ID indicates epilepsy, the road classification ID indicates Tokyo's Metropolitan Expressway, the sensor ID indicates gaze data, the control unit 16 compares the rate of the duration time that are continuously out of the predetermined range of the center part calculated in step S16 with the threshold values Rth1 and Rth2 as shown in FIGS. 18A and 18B, to assess whether or not the epileptic condition or not. If the calculated duration time exceeds the threshold Rth1, or exceeds the threshold Rth2, or a combination of these cases, the control unit 16 assesses that it is in an epileptic condition.

In this manner, the information processing server device 10 functions as an example of the disease-condition assessment means assesses the epileptic condition of the subject depending on duration while the gazing point indicated by the gaze data is out of the center part of a visual field in the traveling direction of the vehicle.

The following specifically describes the case of assessing data of seat pressure distribution.

In case that the disease ID indicates epilepsy, the road classification ID indicates Tokyo's Metropolitan Expressway, the sensor ID indicates gaze data, the control unit 16 compares the area of seat pressure calculated in step S17 with the threshold value Sth as shown in FIG. 21, to assess whether or not the epileptic condition or not. If the calculated area of seat pressure is below the threshold Sth, the control unit 16 assesses that it is in an epileptic condition.

Based on the degree of laterality calculated from the data of the seat pressure distribution, the control unit 16 may assess whether the paralysis is on the left side or the right side.

In this manner, the information processing server device 10 functions as an example of the disease-condition assessment means for assessing the epileptic condition of the subject depending on a change in the size of the seat pressure distribution.

Incidentally, the information processing server device 10 may assess a partial or generalized seizure as disease severity in case of epilepsy.

The information processing server device 10 may combine the operation value, the movement value, and subject sensing value to assess the disease condition. From among operation-quantity data, operation-related data, operation values, movement-quantity data, movement-related data, movement values, subject sensing data, subject sensing-related data, subject sensing values, etc., the information processing server device 10 may assess the disease condition with the most suitable combination of feature values for a predetermined disease.

Incidentally, the information processing server device 10 may assess the disease condition by sequentially acquiring data from the in-vehicle terminal device 30 and other devices. The mobile terminal device 20 or the in-vehicle terminal device 30, as an example of a disease-condition assessment device, may assess the disease condition from the measured data instead of the information processing server device 10. In this case, the control unit 27 of the mobile terminal device 20 or the control unit 37 of the in-vehicle terminal device 30 assesses the disease condition from the measured data.

The information processing server device 10 may assess the disease condition based on the physiological data from the home terminal device 40 and the electronic medical record information from the medical institution server device 50. In particular, on the basis of the physiological data from the home terminal device 40 and the electronic medical record information from the medical institution server device 50, the information processing server device 10 may assess the disease condition.

As thus described, according to this embodiment, by assessing a disease condition of subjects T, such as epilepsy, depending on the relationship between the gaze data indicating the subject T's gazing point measured when the subject T is driving the vehicle V and the driving-characteristic data indicating driving characteristics of the subject T for the vehicle V, it is possible to assess the epileptic condition of the subject T from data that is easy to measure, such as the gaze data and the driving-characteristic data, without special equipment.

In addition, in case that the condition is assessed as epilepsy when the value of the relationship is below a predetermined value, it is possible to easily assess the epileptic condition of the subject T thanks to the value of the relationship.

In addition, in case that the relationship is a degree of dissociation between the gaze data and the driving-characteristic data, it is possible to easily assess the epileptic condition of the subject T thanks to the degree of dissociation.

In addition, in case that the disease condition is epilepsy, when a time for which the degree of dissociation is greater than or equal to a predetermined value is greater than or equal to a predetermined time, the accuracy of assessing the epileptic condition of the subject T can be improved by combining the degree of dissociation with the time for which it is greater than or equal to a predetermined value.

In addition, in case that the driving-characteristic data is at least one of an operation-quantity data of the subject T operating the vehicle V and a movement-quantity data of movement on the vehicle V, it is possible to easily assess the epileptic condition of the subject T thanks to the operation-quantity data, etc.

In addition, in case that the epileptic condition of the subject T is assessed depending on duration while the gazing point indicated by the gaze data is out of the center part of a visual field in the traveling direction of the vehicle V, it is possible to easily assess the epileptic condition of the subject T thanks to this time, duration time calculated from this time, the average time of the duration time, the rate of being located outside the center part, etc.

In addition, according to this embodiment, by assessing an epileptic condition of the subject T depending on a change in the size of the seat pressure distribution on the seating surface where the subject T is seated in the vehicle V, it is possible to assess the epileptic condition of the subject T from data that is easy to measure, such as the seat pressure distribution, without special equipment.

In addition, according to this embodiment, by assessing an epileptic condition of the subject T depending on time that the gazing point, as indicated by the gaze data measured when the subject T is driving the vehicle V, is out of the center part of a visual field in the traveling direction of the vehicle V, it is possible to assess the epileptic condition of the subject T from data that is easy to measure, such as the gaze data, without special equipment.

### Modified Example

The following describes modified examples of disease-condition assessment.

The information processing server device 10 may apply a classifier to the measured data to assess the disease condition. The classifier can be a linear classifier or a nonlinear classifier. It may also be a machine learning classifier where the parameters of the classifier are machine learned. Machine learning includes neural networks, genetic algorithms, Bayesian networks, decision tree learning, and logistics regression.

For example, the information processing server device 10 stores the parameters of the model built by machine learning in the disease assessment DB 12f by performing the machine learning in advance by using the subject sensing data, such as the gaze data, the subject sensing-related data, the subject sensing value, the operation-quantity data, the operation-related data, operation values, the degree of dissociation between the gaze data and the operation-quantity data, etc. Incidentally, the data used for machine learning may be data classified by road classification.

The information processing server device 10 may assess the disease condition by applying a classifier with reference to the disease assessment DB 12f in step S18 to the degree of dissociation between the gaze data and the vehicle driving-characteristic data calculated in step S14.

The information processing server device 10 may assess the disease condition by applying a classifier with reference to the disease assessment DB 12f in step S18 to the operation value and movement value calculated in step S15.

The information processing server device 10 may assess the disease condition by applying a classifier with reference to the disease assessment DB 12f in step S18 to the duration time that the gaze point was continuously located outside the center part calculated in step S16.

The information processing server device 10 may assess the disease condition by applying a classifier with reference to the disease assessment DB 12f in step S18 to the size of seat pressure distribution calculated in step S17.

The information processing server device 10 may assess the disease condition by applying a classifier with reference to the disease assessment DB 12f in step S18 to the calculated operation-related data and the movement-related data.

The information processing server device 10 may assess the disease condition by applying a classifier with reference to the disease assessment DB 12f in step S18 to the data classified depending on the road in step S13.

The information processing server device 10 may assess the disease condition by applying a classifier to the multiple data such as operation-quantity data, movement-quantity data, and subject sensing data with reference to the disease assessment DB 12f.

In case of assessing the disease condition of the subject by machine learning for the operation-quantity data, movement-quantity data, and subject sensing data, etc., the disease condition can be assessed by the pattern of the subject sensing data, the subject sensing values, the waveform of operation-quantity data, operation values, the waveform of movement-quantity data, the movement values, etc.

The mobile terminal device 20 or the in-vehicle terminal device 30 may be equipped with the above classifiers.

In addition, the present invention is not limited to the above embodiments. The above embodiments are merely examples. Any other embodiment that has essentially the same configuration and produces a similar effect as the technical ideas described in the claims of the present invention falls within the scope of the invention.

### REFERENCE SIGNS LIST

10: INFORMATION PROCESSING SERVER DEVICE (DISEASE-CONDITION ASSESSMENT DEVICE)
12: STORAGE UNIT (STORAGE MEANS)
12f: DISEASE ASSESSMENT DATABASE (STORAGE MEANS)
20: MOBILE TERMINAL DEVICE (DISEASE-CONDITION ASSESSMENT DEVICE, TERMINAL DEVICE)
30: IN-VEHICLE TERMINAL DEVICE (DISEASE-CONDITION ASSESSMENT DEVICE, TERMINAL DEVICE)
S: DISEASE-CONDITION ASSESSMENT SYSTEM
T: SUBJECT
V: VEHICLE

1. A disease-condition assessment device comprising:
gaze data acquiring unit for acquiring gaze data indicating a subject's gazing point measured when the subject is driving a vehicle;
driving-characteristic data acquiring unit for acquiring driving-characteristic data indicating driving characteristics of the subject for the vehicle; and
disease-condition assessment unit for being capable of assessing an epileptic condition of the subject depending on the relationship between the gaze data and the driving-characteristic data.

2. The disease-condition assessment device according to claim 1, wherein
the disease-condition assessment unit assesses that the disease condition is epilepsy when the value of the relationship is below a predetermined value.

3. The disease-condition assessment device according to claim 1, wherein
the relationship is a degree of dissociation between the gaze data and the driving-characteristic data.

4. The disease-condition assessment device according to claim 3, wherein
the disease-condition assessment unit assesses that the disease condition is epilepsy, when a time for which the degree of dissociation is greater than or equal to a predetermined value is greater than or equal to a predetermined time.

5. The disease-condition assessment device according to claim 1, wherein
the driving-characteristic data is at least one of an operation-quantity data of the subject driving the vehicle and a movement-quantity data of movement on the vehicle.

6. The disease-condition assessment device according to claim 1, wherein
the disease-condition assessment unit assesses the epileptic condition of the subject depending on duration while the gazing point indicated by the gaze data is out of the center part of a visual field in the traveling direction of the vehicle.

7. The disease-condition assessment device according to claim 1, further comprising
seat pressure distribution acquiring unit for acquiring data of a seat pressure distribution on a seating surface where the subject is seated in the vehicle; and
pressure distribution calculation unit for calculating a size of the seat pressure distribution;
wherein the disease-condition assessment unit assesses the epileptic condition of the subject depending on a change in the size of the seat pressure distribution.

8. A disease-condition assessment method comprising:
a gaze data acquiring step of acquiring gaze data indicating a subject's gazing point measured when the subject is driving a vehicle;
a driving characteristic data acquiring step of acquiring driving-characteristic data indicating driving characteristics of the subject for the vehicle; and
a disease condition assessment step of being capable of assessing an epileptic condition of the subject depending on the relationship between the gaze data and the driving-characteristic data.

9. A non-transitory computer-readable storage medium recording a program for a disease-condition assessment device, for causing a computer to function as:
gaze data acquiring unit for acquiring gaze data indicating a subject's gazing point measured when the subject is driving a vehicle;
driving-characteristic data acquiring unit for acquiring driving-characteristic data indicating driving characteristics of the subject for the vehicle; and
disease-condition assessment unit for being capable of assessing an epileptic condition of the subject depending on the relationship between the gaze data and the driving-characteristic data.

10. A disease-condition assessment system including a terminal device that collects data related to a subject driving a vehicle and a disease-condition assessment device for assessing an epileptic condition of the subject based on the data related to the subject, the system comprising:
the disease-condition assessment device including
gaze data acquiring unit for acquiring gaze data indicating a subject's gazing point measured when the subject is driving a vehicle;
driving-characteristic data acquiring unit for acquiring driving-characteristic data indicating driving characteristics of the subject for the vehicle; and
disease-condition assessment unit for being capable of assessing an epileptic condition of the subject depending on the relationship between the gaze data and the driving-characteristic data.

11. A disease-condition assessment device comprising:
seat pressure distribution acquiring unit for acquiring data of a seat pressure distribution on a seating surface where the subject is seated in the vehicle;
pressure distribution calculation unit for calculating a size of the seat pressure distribution; and
disease-condition assessment unit for being capable of assessing an epileptic condition of the subject depending on a change in the size of the seat pressure distribution.

12. A disease-condition assessment method comprising:
a seat pressure distribution acquiring step of acquiring data of a seat pressure distribution on a seating surface where the subject is seated in the vehicle;
a pressure distribution calculation step of calculating a size of the seat pressure distribution; and
a disease condition assessment step of being capable of assessing an epileptic condition of the subject depending on a change in the size of the seat pressure distribution.

13. A non-transitory computer-readable storage medium recording a program for a disease-condition assessment device, for causing a computer to function as:
seat pressure distribution acquiring unit for acquiring data of a seat pressure distribution on a seating surface where the subject is seated in the vehicle;
pressure distribution calculation unit for calculating a size of the seat pressure distribution; and
disease-condition assessment unit for being capable of assessing an epileptic condition of the subject depending on a change in the size of the seat pressure distribution.

14. A disease-condition assessment system including a terminal device that collects data related to a subject driving a vehicle and a disease-condition assessment device for assessing an epileptic condition of the subject based on the data related to the subject, the system comprising:
the disease-condition assessment device including
seat pressure distribution acquiring unit for acquiring data of a seat pressure distribution on a seating surface where the subject is seated in the vehicle;
pressure distribution calculation unit for calculating a size of the seat pressure distribution; and
disease-condition assessment unit for being capable of assessing an epileptic condition of the subject depending on a change in the size of the seat pressure distribution.

15. A disease-condition assessment device comprising:
gaze data acquiring unit for acquiring gaze data indicating a subject's gazing point measured when the subject is driving a vehicle; and
disease-condition assessment unit for being capable of assessing an epileptic condition of the subject depending on duration while the gazing point indicated the gaze data is out of the center part of a visual field in the traveling direction of the vehicle.

16. A disease-condition assessment method comprising:
a gaze data acquiring step of acquiring gaze data indicating a subject's gazing point measured when the subject is driving a vehicle; and
a disease condition assessment step of being capable of assessing an epileptic condition of the subject depending on duration while the gazing point indicated by the gaze data is out of the center part of a visual field in the traveling direction of the vehicle.

17. A non-transitory computer-readable storage medium recording a program for a disease-condition assessment device, for causing a computer to function as:
gaze data acquiring unit for acquiring gaze data indicating a subject's gazing point measured when the subject is driving a vehicle; and
disease-condition assessment unit for being capable of assessing an epileptic condition of the subject depending on duration while the gazing point indicated by the gaze data is out of the center part of a visual field in the traveling direction of the vehicle.

18. A disease-condition assessment system including a terminal device that collects data related to a subject driving a vehicle and a disease-condition assessment device for assessing an epileptic condition of the subject based on the data related to the subject, the system comprising:
the disease-condition assessment device including
gaze data acquiring unit for acquiring gaze data indicating a subject's gazing point measured when the subject is driving a vehicle; and
disease-condition assessment unit for being capable of assessing an epileptic condition of the subject depending on duration while the gazing point indicated by the gaze data is out of the center part of a visual field in the traveling direction of the vehicle.
